SEQUENCE LISTING (5 sequences)

SEQ ID NO: 1 - DNA, 67 bp, Artificial Sequence
  synthetic nucleic acid
ataagcccttagcgcctattgcgcaaaattttggggtgaaattttcagggtctaggctagctggtag

SEQ ID NO: 2 - DNA, 68 bp, Artificial Sequence
  synthetic nucleic acid
tttaaaagcgcttagctaggcttaaaggtcgtttataagcccttagcgcctattgcgcaaaattttgg

SEQ ID NO: 3 - DNA, 66 bp, Artificial Sequence
  synthetic nucleic acid
gaaattttcagggtctaggctagctggtagctaaacttcgatcgtagctcaggattcccgtttcgg

SEQ ID NO: 4 - DNA, 33 bp, Artificial Sequence
  synthetic nucleic acid
ataagccttagcgcctattgcgcaaaattttgg

SEQ ID NO: 5 - DNA, 70 bp, Artificial Sequence
  synthetic nucleic acid
gaaattttcagggtctaggctagctggtagctaaacttcgatcgatcgtagctcaggattcccgtttcgg

(19) United States
(12) Patent Application Publication
(10) Pub. No.: US 2021/0249102 A1
(43) Pub. Date: Aug. 12, 2021

(54) METHODS FOR COMPARATIVE METAGENOMIC ANALYSIS

(71) Applicant: Arizona Board of Regents on Behalf of the University of Arizona, Tucson, AZ (US)

(72) Inventors: Bonnie L. Hurwitz, Tucson, AZ (US); George S. Watts, Tucson, AZ (US); Illyoung Choi, Tucson, AZ (US); John H. Hartman, Tucson, AZ (US)

(21) Appl. No.: 17/059,993

(22) PCT Filed: May 31, 2019
(86) PCT No.: PCT/US19/34885

§ 371 Date: Nov. 30, 2020

Related U.S. Application Data
(60) Provisional application No. 62/678,947, filed on May 31, 2018.

(51) Int. Cl. G16B 30/00; G16B 50/30; G06K 9/62; G16B 40/00

(52) U.S. Cl. CPC G16B 30/00; G16B 40/00; G06K 9/6223; G16B 50/30

(57) ABSTRACT

Systems and methods for metagenomic analysis are provided. A method of metagenome sequence analysis of two or more samples can include (i) counting the abundance of each k-mer deconstructed from sequencing reads of nucleic acids in each sample, and (ii) using a vector space model to compute the genetic distance between each of the two or more samples according to the abundance of the k-mers. In some embodiments, counting includes (a) constructing a k-mer histogram containing the distribution of k-mers for each sample, and (b) dividing k-mers into partitions having approximately an equal number of k-mers based on the histogram, preparing an inverted index of the k-mers in each partition, and assigning a weight to each k-mer according to its abundance. Method of developing diagnostic and prognostic information using the methods of sequence analysis are also provided.

### CROSS-REFERENCE TO RELATED APPLICATIONS

This application claims benefit of U.S. Provisional Application No. 62/678,947 filed May 31, 2018, which hereby incorporated herein by reference in its entirety.

### STATEMENT REGARDING FEDERALLY SPONSORED RESEARCH

This invention was made with government support under Grant No.

1640775, awarded by NSF and Grant Nos. P30 CA023074 and P30 ES006694, awarded by NIH. The government has certain rights in the invention.

### REFERENCE TO SEQUENCE LISTING

The Sequence Listing submitted as a text file named “UA_18_111_PCT_ST25.txt,” created on May 30, 2019, and having a size of 1,673 bytes is hereby incorporated by reference pursuant to 37 C.F.R § 1.52(e)(5).

### FIELD OF THE INVENTION

The field of the invention generally relates to metagenomic analysis and use thereof for microbial or parasite identification, and infection diagnosis and prognosis.

### BACKGROUND OF THE INVENTION

Microbial communities can be composed of diverse organisms at varied abundances, that change over time given microbe-microbe interactions and ecosystem processes. Capturing the details of these interactions remains elusive given that the majority of microbes are unculturable (Yooseph, et al., PLoS Biol., 5: e16 (2007); Sunagawa, et al., Science, 348(6237):1261359, doi:10.1126/science.1261359 (2015)). Metagenomics, a method to sequence DNA/RNA directly from an environment, offers a path forward to analyze the complete genetic repertoire of a microbial or parasitic community—including both known and new species. In the last decade, the cost of sequencing has decreased more than a million-fold, leading to a rapid influx of metagenomic data from diverse environments that promises insight into new organisms and ecosystem function. Yet, with the great wealth of sequencing data comes great responsibility in analyzing massive and ever-increasing data sets. To discover the role of new microbes in varied environments and conditions, these large-scale metagenomes need to be compared against one another to examine environmental characteristics that define microbial or parasitic community composition.

Amplicon sequencing of the 16S ribosomal RNA (rRNA) gene has been fundamental to addressing questions about bacterial diversity in environmental samples. The 16S rRNA gene is highly conserved, but contains hypervariable regions that can be used to distinguish bacteria at the genus level Amplicon datasets are often large, and can be rapidly reduced into clusters of operational taxonomic units (OTUs) to quantify the relative abundance of bacteria in a sample Amplicon datasets are useful for surveying bacterial diversity at the genus-level, but provide no information about the function of bacteria in a given environment. Whole genome shotgun (WGS) sequencing offers increased resolution at the species and subspecies level and can be used to infer both taxonomy and function. Yet, the resulting data are often massive and time consuming to analyze using traditional sequence comparison methods.

Recently, fast k-mer based algorithms have been developed to classify metagenomic reads against known microbial genomes at remarkable throughput and speed. Specifically, Centrifuge (Kim, et al., “Centrifuge: rapid and sensitive classification of metagenomic sequences,” bioRxiv, p. 054965, doi:10.1101/054965 (2016), CLARK (CLAssifier based on Reduced K-mers) (Ounit, et al., BMC Genomics, 16:236 (2015)), USEARCH (Edgar, et al., BMC Bioinformatics, 26:2460-2461 (2010)), KRAKEN (Wood, et al., Genome Biol, 15:R46 (2014)), and NBC (Naive Bayes Classifier) (Rosen, et al., Adv Bioinformatics, 2008:205969, doi:10.1155/2008/205969 (2008)) rapidly identify microbial or parasitic species present in a metagenome using genetic composition-based methods. In each case, frequency profiles of k-mers from microbial genomes are built to rapidly assign reads to genomes in a reference database (Ounit, et al., BMC Genomics, 16:236 (2015); Wood, et al., Genome Biol, 15:R46 (2014); Rosen, et al., BMC Bioinformatics, 27:127-129 (2011); Bazinet, et al., BMC Bioinformatics, 13:92 (2012)). Although these methods are fast and outperform alignment-based methods, building k-mer frequency profiles for the reference genomes requires large-amounts of memory (>128 GB of RAM) (Ounit, et al., BMC Genomics, 16:236 (2015)).

To compare samples based on the complete genomic content (including known and unknown organisms simultaneously) reference-free k-mer based clustering approaches can be employed such as UCLUST (Edgar, et al., BMC Bioinformatics, 26:2460-2461 (2010)), Jellyfish (Marçais, et al., Bioinformatics, 27:764-770 (2011)), and GenomeTools (Tallymer) (Gremme, et al., IEEE/ACM Trans Comput Biol Bioinform, 10:645-656 (2013)). These approaches rely on three core tenets of k-mer-based analytics: (i) closely related organisms share k-mer profiles and cluster together, making taxonomic assignment unnecessary (Dubinkina, et al., BMC Bioinformatics, 17:38 (2016); Teeling, et al., BMC Bioinformatics, 5:163 (2004)), (ii) k-mer frequency is correlated with the abundance of an organism (Wu, et al., J Comput Biol, 18:523-534 (2011)), and (iii) k-mers of sufficient length can be used to distinguish specific organisms (Fofanov, et al., Bioinformatics, 20:2421-2428 (2004)). Yet because each of these tools relies on a single server for computation, local resources such as memory and disk are quickly consumed by all-vs-all sequence comparisons and cannot scale for large studies. Other methods have been developed that reduce the dimensionality of metagenomes by creating unique k-mer sets and computing the genetic distance between pairs of metagenomes, such as Compareads (Maillet, et al., BMC Bioinformatics, 13 Suppl 19:S10 (2012)), Commet (Maillet, et al., IEEE International Conference on Bioinformatics and Biomedicine (BIBM); 94-98 (2014)), MetaFast (Ulyantsev, et al., Oxford Univ Press, 32:2760-2767 (2016)), and Mash (Ondov, et al, Genome Biol., 17:132 (2016)). For example, Mash indexes samples by unique k-mers to create size-reduced sketches, compares these sketches using the min-Hash algorithm (Chum, et al., “Near Duplicate Image Detection: min-Hash and tf-idf Weighting” BMVC, (2008)), and computes a genetic distance using Jaccard similarity. The result is a 7000-fold compression of input sequence data and fast pairwise comparison of samples (Ondov, et al, Genome Biol., 17:132 (2016)). Yet, the tradeoff for speed is that samples are reduced to a subset of k-mers (1k by default) and lack information on k-mer abundance in the samples. Thus, Mash only accounts for genetic distance between samples (or genetic content in microbial communities) without considering abundance (dominant vs rare organisms in the sample) that is central to microbial ecology and ecosystem processes.

Recently, SIMKA (Benoit, et al., PeerJ Comput Sci. PeerJ Inc.; 2:e94 (2016)) was developed to compute a distance matrix between metagenomes using all k-mers within a sample. SIMKA executes analyses on a high-performance compute cluster (HPC) by dividing the input datasets into abundance vectors from subsets of k-mers, then rejoining the resulting abundances in a cumulative distance matrix. SIMKA also provides the user with various ecological distance metrics to let the user choose the metric most relevant to their analysis. Because some distance metrics are complex (such as Jensen) and scale quadratically as more samples are added computational time and complexity can vary based on the distance metric used (Benoit, et al., PeerJ Comput Sci. PeerJ Inc.; 2:e94 (2016)).

Thus, there remains a need for solutions that improve the speed and efficiency of metagenomic data analysis without a corresponding loss in important information such as identification accuracy or organism abundance within a population.

It is an object of the invention to provide materials, methods, systems and other solutions for use in metagenomics analysis.

### SUMMARY OF THE INVENTION

Systems and methods for metagenomic analysis are provided. A method of metagenome sequence analysis of two or more samples can include (i) counting the abundance of each k-mer deconstructed from sequencing reads of nucleic acids in each sample, and (ii) using a vector space model to compute the genetic distance between each of the two or more samples according to the abundance of the k-mers. In some embodiments, counting includes (a) constructing a k-mer histogram containing the distribution of k-mers for each sample, and (b) dividing k-mers into partitions having approximately an equal number of k-mers based on the histogram, preparing an inverted index of the k-mers in each partition, and assigning a weight to each k-mer according to its abundance.

In some embodiments, the inverted index is indexed by k-mer sequence and includes a canonical representation of each k-mer, an identifier of the samples that contain that k-mer, and its frequency in each sample. The canonical representation of each k-mer can be, for example, either the forward form or the reverse complement form of the k-mer depending on which is first alphabetically.

The vector space model can include assigning each sample a vector, wherein each dimension of each sample's vector corresponds to a unique k-mer and wherein the length and the angle of the vector relates to the abundance of the k-mer and indicates the weight given to the corresponding k-mer in the inverted index. In some embodiments, the genetic distance between samples is determined using the cosine of the angles (i.e., cosine similarity) between the vectors of the two or more samples.

The methods are typically computer implemented, and can employ a Hadoop platform using Hadoop tasks to execute the method in a balanced fashion over two or more computers. For example, in some embodiments, a computer implemented method of metagenome sequence analysis of two or more samples includes (i) counting the abundance of each k-mer deconstructed from sequencing reads of nucleic acids in each sample including (a) constructing a k-mer histogram containing the distribution of k-mers deconstructed from sequencing reads of nucleic acids in each sample, (b) dividing k-mers into partitions having approximately an equal number of k-mers based on the histogram, preparing an inverted index of the k-mers in each partition and assigning a weight to each k-mer according to its abundance, and (ii) using a vector space model to compute the genetic distance between each of the two or more samples according to the abundance of the k-mers, wherein the vector space model comprises assigning each sample a vector, wherein each dimension of each sample's vector corresponds to a unique k-mer and wherein the length and the angle of the vector relates to the abundance of the k-mer and indicates the weight given to the corresponding k-mer in the inverted index. (i), (ii), or a combination thereof can be executed using Map and Reduce task functions on a Hadoop cluster of two or more computers. (i), (ii), or a combination thereof can be executed using Spark task functions optionally on a Hadoop cluster of two or more computers. In some embodiments, at least the counting is distributed over the two or more computers of the Hadoop cluster, and in some embodiments the workload is balanced among the computers in the cluster. For example, in some embodiments, balancing the workload includes splitting sequencing files into data blocks at the block boundary. In some embodiments, (i) and/or (ii) are carried out in real-time.

Sequences can be from samples with known composition (i.e., the microbes within the sample are known), unknown or incomplete composition (i.e., one or more of the microbes in the sample are unknown), or a combination thereof. Samples include samples from a biological site, environmental samples, industrial samples, water samples, soil samples, air samples, etc. Samples can be biological samples from a subject in need of diagnosis or prognosis (e.g., a sample from an undiagnosed or prognosed infection). Such samples are typically unknown or incomplete. Samples can also be from previous sequencing, and optionally analysis, of known organisms (e.g., known databases, sequence deposits, etc.), or samples in which a clinical outcome is known (e.g., a sample from an infection that was successfully or unsuccessfully treated). In some embodiments, sequencing reads are in the form of a set of sample files, each of which contains the sequence data for a single sample.

The methods can include using the determination of genetic distance between the two or more samples to identify taxonomic differences between two or more samples, determine the taxonomy of one or more of the samples, or a combination thereof. The methods can also include using the genetic distance between the two or more samples to identify functional differences between two or more samples, determine a function of one or more of the samples, or a combination thereof.

Diagnostic and prognostic methods are also provided. For example, a method of diagnosing an infection in a subject in need thereof can include metagenome sequence analysis, wherein at least one of the samples is a biological sample from a subject, determining the taxonomy or a function of the biological sample, and diagnosing the subject based on the taxonomy or function.

A method of prognosing an infection in a subject in need thereof can include metagenome sequence analysis, wherein at least one of the samples is a biological sample from a subject, and at least one of the samples is a known clinical sample from a clinical subject's infection, and the result or outcome of treatment of the clinical subject is known, prognosing the subject based on the genetic distance between the subject's sample and the clinical sample.

Any of the methods can further include treating the subject for a disease or disorder.

In some embodiments, the method is used to identify biomarkers. For example, in some embodiments, nucleic acid sequences are identified that are unique to a microorganism causing or contributing to the disease or disorder and/or specific for the disease or disorder.

Systems for metagenomic analysis leading to diagnostic and prognostic information are also provided. In some embodiments, the systems include one or more processors and one or more non-transitory computer readable storage media storing computer readable instructions that when executed by the one or more processors cause the processors to perform the disclosed method. In some embodiments, the system further includes two or more additional computers that carries out the method under the direction of a first controlling computer. In some embodiments, the system includes two or more computers in a Hadoop cluster.

In some embodiments a client (e.g., a first user) requests to establish an electronic relationship with the system. In some embodiments, the client provides the input (e.g., the samples, or sequences therefrom) for metagenomics analysis to provide diagnostic and/or prognostic information by the system. In some embodiments, the system performs a method of metagenomics analysis after receiving an input from a client.

Non-transitory computer-readable media for metagenomics analysis is also provided. The non-transitory computer-readable media can store instructions that when executed cause a computer to perform the disclosed methods of metagenomic analysis and generation of diagnostic and/or prognostic information.

### BRIEF DESCRIPTION OF THE DRAWINGS

FIG. 1A is an illustration of an exemplary workflow of an exemplary embodiment (i.e., Libra) of the disclosed analytical methods. The illustration shows three MapReduce jobs—1) k-mer histogram construction, 2) inverted index construction and 3) distance matrix computation (scoring). K-mer histograms are first constructed for input samples to balance workloads over the Hadoop cluster in following jobs. Inverted indices are constructed per a group of samples in parallel by partitioning k-mer ranges. An index chunk is produced from each partition and an inverted index is comprised of multiple index chunks. In scoring, partial contribution is computed within a partition and accumulated to the final distance matrix. FIG. 1B is a flow diagram showing a workflow for k-mer index entry preparation. FIG. 1C is a flow diagram showing a workflow of kmer index construction. FIG. 1D is an illustration of k-mer matching. FIG. 1E is flow diagram illustrating k-mer counting and frequency determination.

FIG. 2 is an illustration of k-mer-level sequence data splitting, illustrated with five reads: Read 1 (SEQ ID NO:1), Read 2 (SEQ ID NO:2, Read 3 (SEQ ID NO:3), Read 4 (SEQ ID NO:4), and Read 5 (SEQ ID NO:5).

FIG. 3A is a histogram showing the distribution of raw k-mers in a sample. FIG. 3B is a histogram showing the distribution of canonical k-mers in a sample. (POV_M.Fall.I.42m_reads.fa, Pacific Ocean Viromes dataset, k=12)

FIG. 4 is a histogram showing k-mer range partitioning based on k-mer frequencies.

FIGS. 5A and 5B are line graphs showing changes of the imbalance (5A: the number of imbalanced partitions (imbalance threshold=1%)) and the size of k-mer histogram for different k-mer prefix lengths (5B: the size of k-mer histogram). (POV_M.Fall.I.42m_reads.fa of Pacific Ocean Viromes dataset, k=20)

FIG. 6 is an illustration of histogram-based input-splitting in distance matrix computation.

FIGS. 7A-7B are bar graphs showing analysis of artificial metagenomes using MASH, SIMKA and Libra. FIG. 7A illustrates the distance to staggered mock community artificial metagenome composed of 10 million reads (mock1 10M), for artificial metagenomes of same community sequenced at various depth. Artificial metagenomes (454 sequencing) were obtained using GemSim and the known abundance profile of the staggered mock community (see Table 2). In order to mimic various sequencing depth, the artificial metagenomes were generated at 0.5, 1, 5 or 10 million reads (noted bars from left-to-right for each method mock1 10M; mock1 0.5M; mock1 1M; mock1 5M; mock1V2 10M). The distances between the 4 artificial metagenomes and a 10 million read artificial metagenome (mock1 10M) were computed using MASH, SIMKA (Jaccard and Bray-Curtis distance) and Libra (natural weighting). FIG. 7B illustrates the distance to staggered mock community artificial metagenome (mock 1), for artificial metagenomes from increasingly distant communities. The mock 1 relies on the known abundance profile from the staggered mock community. The mock 2 community profile was obtained by randomly inverting 3 species abundance from mock 1 profile. The mock 3 profile was obtained by randomly inverting 2 species abundances from mock 2 profile. Finally, mock 4 profile was obtained by adding high abundance archaeal genomes not present in any the other mock communities. Artificial metagenomes (454 sequencing) were generated using GemSim at 10 million reads. The distance between the mock 1 community to mock2, mock3, mock4 and a replicate community (mock1 V2) (bars from left-to-right) for each computing method: MASH, SIMKA (Jaccard and Bray-Curtis distance) and LIBRA (Natural and Log weighting).

FIGS. 8A-8C are heat maps showing analysis of binary microbial mixtures using MASH, SIMKA and Libra. Three binary mixtures of bacterial species across a six log range of dilution were considered: E. coli versus S. saprophyticus (8A); S. saprophyticus versus S. pyogenes (8B) and E. coli versus S. flexneri (8C). Sample-to-sample distances were computed using MASH (first column), SIMKA using either the presence/absence Jaccard distance (second column), the bray-curtis distance (third column) and the Jensen distance (third column), and Libra (fourth column) Heat maps illustrate the pairwise dissimilarity between samples, scaled between 0 and 1. FIG. 8D is a heat map showing a comparison of SIMKA distances for the analysis of binary microbial mixtures. Abundance-based distances are computed using LIBRA on three binary mixtures of bacterial species across a six log range of dilution were considered: E. coli versus S. flexneri (first line); E. coli versus S. saprophyticus (second line) and S. saprophyticus versus S. pyogenes (third line).

FIG. 9A-9D are heat maps showing clustering of HMP 16s and WGS samples using MASH and Libra. 48 Human metagenomic samples from the HMP projects clustered by Mash or Libra from 16s sequencing runs (9A-9B) and the corresponding whole genome shotgun sequencing runs (9C-9D). The samples were clustered using Ward's method on their distance scores. Heat maps illustrate the pairwise dissimilarity between samples, scaled between 0 and 1. FIG. 9E-9F are heat maps showing comparison of MASH and Libra for the analysis and clustering of HMP assemblies. Sample to sample distance was computed on 48 HMP assemblies using MASH (9E) or Libra (9F). The samples were clustered using Ward's method on their distance scores. A key below the heatmaps colors the samples by body sites.

FIG. 10 is an illustration visualizing the genetic distance among marine viral communities using Libra. Distance computed from 43 TOV from the 2009-2012 Tara Oceans Expedition. Lines (edges) between samples represent the similarity and are colored and thickened accordingly. Lines with insignificant similarity (less than 30%) are removed. Each of the sample names are color coded by Longhurst Province. Inner circles show temperature ranges. Sample names show the temperature range, station, and depth as indicated on the legend.

FIG. 11 is line graph showing map task durations during inverted index construction.

FIG. 12 is a line graph showing reduce task durations during inverted index construction.

FIG. 13 is a line graph showing index chunk sizes.

FIG. 14 is a line graph showing map task durations during distance matrix computation.

FIG. 15 is an illustration of an exemplary generalized computer network arrangement.

FIG. 16 is a flow chart showing an exemplary workflow for identification and prognosis utilizing metagenomic analysis.

FIG. 17 is a schema of the sweep line algorithm in scoring. A sweep line moves from the first dimension to the last (left to right). At every dimension containing k-mers (dots), an output record is produced from the weights of the k-mers (based on k-mer abundance) on the sweep line.

### DETAILED DESCRIPTION OF THE INVENTION

### I. Methods of Sequence Data Analysis

Scientific collaboration is increasingly data driven given large-scale next generation sequencing datasets. It is now possible to generate, aggregate, archive, and share datasets that are terabytes and even petabytes in size. Scalability of a system is becoming a vital feature that decides feasibility of massive omics' analyses. In particular, this is important for metagenomics where patterns in global ecology can only be discerned by comparing the sequence signatures of microbial communities from massive ‘omics datasets, given that most microbial genomes have not been defined.

Current algorithms to perform these tasks run on local workstations or high-performance computing architectures that cannot scale. The methods disclosed herein can be implemented via cloud-based resource for comparative metagenomics that can be broadly used by scientists to analyze large-scale shared data resources.

Metagenomics typically includes comparing DNA samples collected from the environment. DNA is a double helix structure composed of two strands that are complements of each other. A strand is a sequence of four characters, ‘A’, ‘T’, ‘G’ and ‘C’, representing the four nucleobases adenine, thymine, guanine and cytosine, respectively. ‘A’-‘T’ and ‘G’-‘C’ are paired in the complementary strands. Each strand has an orientation, i.e. ‘5’-end to 3′-end, and complementary strands have opposite orientations.

The sequence of A, T, G, and C nucleobases in a strand of DNA is read by sequencing machines, and is called a read. There is a limit, however, to the number of sequential nucleobases that current sequencing technology can produce, which given current technologies is length of a few hundred nucleobases.

When computing the entire DNA sequence for a particular organism these limitations are overcome by producing many overlapping reads of the DNA, then stitching them together to produce the entire DNA sequence. In contrast, in metagenomics the reads are produced from all the DNA in the environmental sample, regardless of which organism they originated from. The sequence can be read in two ways, forward and reverse-complement, each representing a particular strand of DNA.

A common way of analyzing the DNA in a metagenomic sample is through the statistical analysis of k-mers. A k-mer—also known as an n-gram—is a substring of length k, typically produced from every offset in a string. In a string in length kL, there exists total (L−k+1) k-mers. Also, there can exist at most C^{k} distinct k-mers in a string composed of C distinct characters. k-mers are used in many fields, such as computational linguistics, to retrieve similar documents by comparing the k-mer composition of documents.

Measuring the distance—or similarity/dissimilarity—between samples is an important process in metagenomics. By looking at the distance between samples from different time or environments, correlations can be found and bring new insights for further research.

Traditionally, the distance of metagenomic samples has been measured by comparing composition of known organisms in samples. This method requires mapping of sequence data to known organisms using reference databases. However, this less effective in large-scale metagenomics studies because a high portion of sequence data is derived from unknown organisms.

The k-mer-based reference-free distance computation is a widely accepted method of comparing genomic signatures—based on k-mer composition which includes both known and unknown organisms—in large scale metagenomics studies. The method relies on three core tenets: (i) closely related organisms share k-mer profiles and cluster together, making taxonomic assignment unnecessary (Dubinkina, et al., BMC Bioinformatics, 17(1) 38 (2016); Teeling, et al., BMC Bioinformatics, 5:163 (2004)), (ii) k-mer abundance is correlated with the abundance of an organism (Wu, et al., Journal of computational biology: a journal of computational molecular cell biology 18, 3: 523-534 (2011)), and (iii) k-mers of sufficient length can be used to distinguish specific organisms (Fofanov, et al., Bioinformatics, 20(15) 2421-2428 (2004)). This method offers high performance and precise results compared to traditional methods of analyzing the known organisms only in large scale metagenomic studies.

The analysis is performed in two steps: (i) k-mer counting and (ii) distance matrix computation. First, the abundance of each k-mer is counted in each sample, and indices are constructed to provide an efficient means of determining k-mer abundances in each sample. Second, the indices are used to compute a distance between each pair of samples, such that the distance is inversely proportional to the similarity between the samples. Historically, the distance was typically measured based on the abundance of k-mers by using various statistical functions, such as Jaccard, Bray-Curtis, and Jensen. The disclosed methods typically utilized Cosine Similarity as a distance metric for comparing genomic datasets.

There are two known difficulties in the k-mer-based distance computation related to the large number of: (i) k-mers produced and (ii) pairwise comparisons. First, approximately 500 million k-mers are produced in a one GB sample. This means that today's metagenomic datasets contain hundreds of billions of k-mers (see Table 1). Also, in practice, k is at least 20 base pair (bp), so that the k-mers match a particular organism and are not random. For example, if k is 20 bp, there are 4²⁰ possible k-mers. Considering the scale of today's metagenomic datasets, such as the Tara Ocean Viromes (TOV) dataset (Brum, et al., Science, 348:6237 (2015)) shown in Table 1, processing this enormous number of k-mers is challenging and requires massive compute and storage resources.

Second, the number sample pairs increases quadratically as the number of samples in a dataset increases. There are n×(n−1)/2 n×(n−1)/2 sample pairs in a dataset containing n samples, e.g. there are 903 pairs of samples in a dataset with 43 samples. Computing the distance of each pair of samples individually is too computationally expensive to be practical.

TABLE 1
Statistics of the Tara Ocean Viromes (TOV) dataset.

| Number of samples | Size of dataset | Total number of reads |
|---|---|---|
| 43 | 551.6 GB | 4,194,402,268 |

| Total number of base pair | Total number of 20-mers |
|---|---|
| 403,891,365,808 | 324,197,722,716 |

There are several k-mer counting tools available, such as KMC2 (Deorowicz, et al., Bioinformatics, 31(10) 1569-1576) and DSK (Rizk, et al., Bioinformatics, 29 (5) 652-653 (2013)). These tools are optimized to count k-mers in a sample efficiently without requiring huge RAM (Perez, et al., Journal of computational biology: a journal of computational molecular cell biology, 23(4) 248-255) in a machine. Their ideas are mainly about efficient use of disks to overcome insufficient RAM and compression of k-mer data to reduce the storage required. However, these tools are not suitable for large-scale metagenomics study because they need an increasingly powerful machine to keep pace with the every-growing size of metagenomic datasets. Also, these tools merely count k-mers, requiring the distance computation to be performed in a separate computation.

Mash (Ondov, et al., Genome Biology, 17(1) 132 (2016)) is a distance computation tool. It solves the scalability issue using subsampling. Mash creates size-reduced sketches of k-mer composition of samples using the MinHash algorithm (Chum, et al., BMVC (2008)), compares these sketches and computes a genetic distance using Jaccard similarity. Yet, the tradeoff for speed is that samples are reduced to a subset of k-mers (1k by default) and lack information on k-mer abundance in the samples. Thus, Mash only accounts for genetic distance between samples without considering abundance (dominant vs rare organisms in the sample) that is central to microbial ecology and ecosystem processes.

A distance computation tool, SIMKA (Benoit, et al., Computer Science, 2: e94 (2016)), runs on an HPC cluster. It solves the scalability issue using distributed computing. However, SIMKA has three important limitations. First, tasks for the k-mer count are distributed by sample. This can cause workload imbalance when the samples vary in size or there are fewer samples than machines in the cluster. Second, the k-mer abundances produced by different machines must be aggregated, involving large amounts of disk I/O. Third, SIMKA requires specific schedulers, such as Sun Grid Engine (SGE).

Oracle Grid Engine, previously known as Sun Grid Engine (SGE), CODINE (Computing in Distributed Networked Environments) or GRD (Global Resource Director), was a grid computing computer cluster software system (otherwise known as a batch-queuing system). Grid Engine is typically used on a computer farm or high-performance computing (HPC) cluster and is responsible for accepting, scheduling, dispatching, and managing the remote and distributed execution of large numbers of standalone, parallel or interactive user jobs. It also manages and schedules the allocation of distributed resources such as processors, memory, disk space, and software licenses.

Improved analytic methods for use in comparative genomics are provided. A computer implement embodiment exemplified in the working Examples below is referred to herein as Libra.

A. Overview

Methods of analyzing genetic sequences, for example comparative metagenomics are provided. In some computer implemented embodiments, the methods are carried out using a data processing algorithm such as a MapReduce algorithm and/or a Spark algorithm. The methods provide a means of performing all-vs-all sequence analysis on large-scale data sets, and uses a vector space model (Salton, et al., Communications of the ACM, 18(11) 613-620) to consider genetic distance and microbial abundance simultaneously. The methods provide accurate, efficient, and scalable computation for comparative metagenomics that can be used to discern global patterns in microbial ecology, and utilized for methods of diagnosis, prognosis, and treatment. The method can be generalized to any all-vs-all sequence comparisons using raw reads from next-generation sequence (NGS) technologies to rapidly compute taxonomic and functional differences in samples from human health to the environment. These analyses are fundamental for determining how samples cluster, which can inform follow up analyses on the underlying biological mechanisms and microbial communities that drive the clustering.

MapReduce is a high-level programming abstraction that greatly simplifies the development and deployment of new analytical tools (Dean, et al., Communications of the ACM, 51(1) 107-113 (2008)). Programmers implement these tools in terms of “map” and “reduce” functions. The computation is most typically implemented on the Hadoop platform using Hadoop tasks.

Apache Spark has as its architectural foundation the resilient distributed dataset (RDD), a read-only multiset of data items distributed over a cluster of machines, that is maintained in a fault-tolerant way. Spark and its RDDs were developed in response to limitations in the MapReduce cluster computing paradigm, which forces a particular linear dataflow structure on distributed programs: MapReduce programs read input data from disk, map a function across the data, reduce the results of the map, and store reduction results on disk. Spark generalizes MapReduce's functionality to allow non-linear dataflows. Datasets are stored in RDDs. A “transformation” produces a new RDD from existing one, while an “action” computes a value from an RDD. A transformation is therefore a generalization of “map” while an action is a generalization of “reduce”. For performance RDDs are stored in main memory.

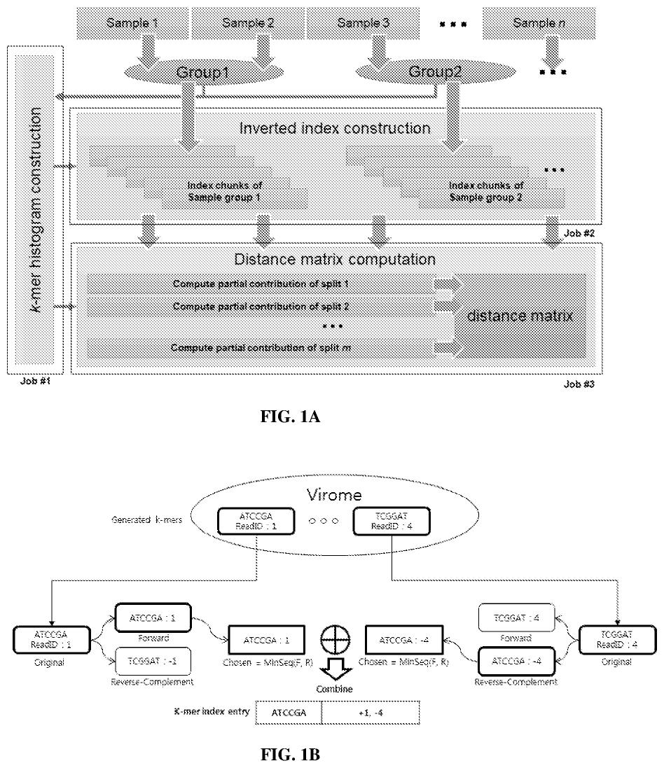

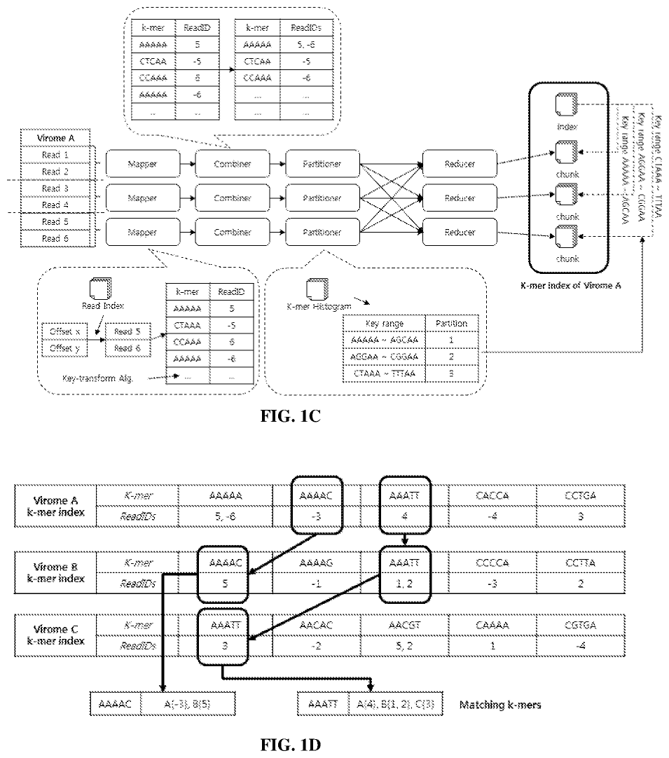

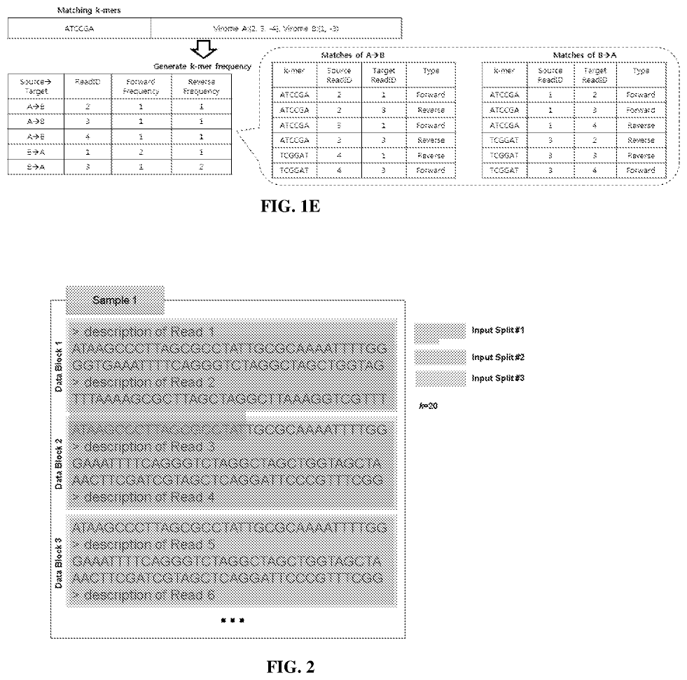

The disclosed methods can be implemented using three different MapReduce or Spark jobs—1) k-mer histogram construction, 2) inverted index construction (k-mer counting), and 3) distance matrix computation (scoring). FIGS. 1A-1E show exemplary workflows.

First a k-mer histogram containing the distribution of k-mers for each sample is constructed. In the MapReduce Map phase, a separate Map task is spawned for each data block of the input sample files. The Map tasks calculate the k-mer histograms for the blocks in parallel. In the Reduce phase, a Reduce task is spawned to aggregate the partial k-mer histograms produced by Map tasks to produce a final histogram. In Spark the kmer-histograms are created by a transformation of the sample RDDs into a histogram RDD.

The inverted index construction can be performed in parallel. In the MapReduce Map phase, a separate Map task can be spawned for every data block in the input sample files. Each Map task generates the k-mers from the sequences stored in a data block, then passes them to the Reduce tasks. In the Reduce phase, the k-mer histograms computed in the first phase are used to partition the k-mer space. A separate Reduce task is spawned for each partition and in the shuffle step a custom Partitioner routes the produced k-mers to the proper Reduce tasks based on their partitions. Each Reduce task then counts the k-mers it receives and produces an index chunk. As a result, each index chunk is stored as a separate file in the Hadoop MapFile (Zuanich, “Hadoop I/O: Sequence, Map, Set, Array, BloomMap Files,” Cloudera Engineering Blog (2011)) format. The MapFile is well-suited for the disclosed methods as it is designed to store key-value pairs in key order, allowing for binary search of the keys. In Spark a transformation of the sample RDDs creates the inverted index RDD.

In the distance matrix phase the work is distributed based on the k-mer histograms. The k-mer histogram files for the input samples are loaded and are used to split the k-mer space. In MapReduce a separate Map task is spawned for each split and performs the computations in parallel. As a result, each task produces an output file containing partial contributions to the distance matrix. At the end of the job, the partial contributions from the files are merged and the complete distance matrix is produced. In Spark the k-mer histogram RDDs for the input samples are used to split the k-mer space, after which transformations of the sample RDDs produce the partial contributions to the distance matrix RDD, after which an aggregation transformation is used to produce the complete distance matrix.

B. Task Load Balancing

There are several factors that can cause task workload imbalance including (i) the sizes of the samples and (ii) uneven k-mer distributions. These factors can be addressed by the disclosed methods to provide balanced workloads among the tasks and avoid bottlenecks.

1. Splitting the Input Dataset

The input is typically composed of a set of sample files, each of which contains the sequence data for a single sample. A sample file can be in, for example, FASTA or FASTQ format, which are text-based and contain the reads obtained from the sample. In bioinformatics, FASTA format is a text-based format for representing either nucleotide sequences or peptide sequences, in which nucleotides or amino acids are represented using single-letter codes. The format also allows for sequence names and comments to precede the sequences. The format originates from the FASTA software package, but has now become a standard in the field of bioinformatics. The simplicity of FASTA format makes it easy to manipulate and parse sequences using text-processing tools and scripting languages like R, Python, Ruby, and Perl. FASTQ format is a text-based format for storing both a biological sequence (usually nucleotide sequence) and its corresponding quality scores. Both the sequence letter and quality score are each encoded with a single ASCII character for brevity. It was originally developed at the Wellcome Trust Sanger Institute to bundle a FASTA sequence and its quality data, but has recently become the de facto standard for storing the output of high-throughput sequencing instruments such as the Illumina Genome Analyzer.

Reads can be of variable length and include multiple sections. For example, FASTA has 2 sections: description line and sequence. The description line of FASTA format always starts with a symbol “>” to indicate the start of a read. New line character “\n” is a delimiter for sections.

Counting k-mers in a large-scale dataset requires so much storage for intermediate data that it can exceed the storage capacity of a cluster. The disclosed methods can solve this problem by grouping samples together to count k-mers per-group, rather than per-sample. Samples in a dataset should be grouped properly in respect to factors such as (i) the size of a group should not be so large as to exceed the storage capacity of the cluster and (ii) the size of a group should not be so small as to underutilize cluster resources. There is no single value for the size of a group that satisfies all because it varies depending on the cluster. Therefore, users generally adjust the value to achieve good performance.

The tasks of counting k-mers in a group can be distributed over a cluster. There are several approaches to distributing the tasks: file-level, read-level, and k-mer-level.

Distributing the tasks by sample file (file-level) is an approach to distributing the tasks over a cluster. However, this approach can cause workload imbalance when the samples are not the same size. This happens because some samples have been sequenced more deeply and therefore have more reads. In addition, as the size of samples grows, more powerful machines will be needed. For example, the largest sample in the Tara Ocean Viromes (TOV) dataset is 2× bigger (22.6 GB) than the smallest (8.7 GB) and an individual sample is large (AVG. 12.8 GB). Considering the number of k-mers produced and the workload in k-mer counting, assigning a whole file to a task is inefficient.

Distributing the tasks at the block-level is the default for Hadoop. Input samples are split based on file system data blocks, i.e., HDFS (Shvachko, et al., IEEE 26th Symposium on Mass Storage Systems and Technologies (MSST), 1-10 (2010)). This approach is preferred in Hadoop for two reasons: (i) it limits the difference in workload between partitions to block size that is configurable (by default, 64 MB) and (ii) a task can be assigned a data block on the same machine.

Splitting input samples at the block-level arises a concern, however: a read can span a block boundary. This can be solved by adjusting the task range to align to the read boundary. However, this can cause load imbalances because the reads are of varying size.

The disclosed methods solve this problem by k-mer-level splitting, i.e., splitting the read at the block boundary (FIG. 2). An assumption is made: the description line in a read does not exceed D bytes (by default, D=10240). The splitting algorithm works as follows. First, samples are split based on data blocks. Second, the reader module of a task checks if its block begins in the middle of the sequence section of a read. The reader module looks backward at most D bytes for the beginning of the read. If the start of a read is found but a new line is not detected, this indicates that a descriptor line spans on the boundary. In this case, the module ignores the line because description is not used in the k-mer counting. If the start of a read is found and a new line is detected, this indicates that sequence spans on the boundary. Third, the module breaks the sequence at the boundary. This causes a difficulty with k-mers that span the split, therefore the reader module also reads k−1 bytes beyond the end of the current block to capture these k-mers.

2. k-Mer Counting and Partitioning

In the Map phase of the MapReduce implementation, each Map task generates k-mers from sequences in an assigned data block and produces a record containing k-mer, FileID and abundance of the k-mer. k-mers in the records are stored in the canonical form—either the forward or the reverse-complement form of the k-mer, whichever comes first lexicographically. This is a technique to find the forward-strand and the reverse-complement-strand of k-mers using a single search. FileID can be a number assigned in the beginning of the computation to avoid the space required to store long filenames. The k-mers produced by the Map task are sent to the Reduce tasks for aggregation. Each Reduce task then aggregates and counts the k-mers that it receives and produces an index chunk. As a result, each index chunk is stored as a separate file. See, e.g., FIG. 1B-1C.

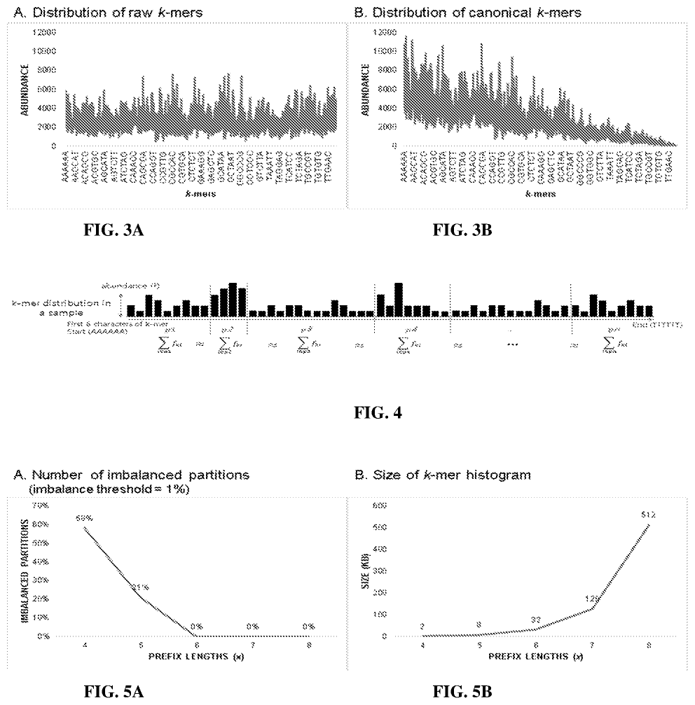

The partitioner partitions the k-mers produced by the Map tasks and assigns them to the proper Reduce tasks for aggregation. Preferably k-mers are partitioned so that the workloads on the reducers are balanced. There are several approaches to partition records, but they may not lead to balanced workloads. Partitioning records by dividing the k-mer space into equal ranges is one approach. However, this can cause the workload imbalance because the distribution of k-mer is not uniform in metagenomic datasets as shown in FIG. 3A. After taking the canonical form of k-mers, the distribution becomes highly skewed as shown in FIG. 3B. This is because the canonicalization will make many of k-mers starting with “T” or “G” to be reverse-complemented. Therefore, the equal range partitioning is not preferred.

Partitioning k-mers by their hash is a widely-accepted approach for dealing with non-uniform distributions in Hadoop keys, however, this approach is typically avoided in the disclosed methods because the resulting inverted indices are not reusable because the hashing makes k-mers unordered across index chunks. Partitioning based on hashing also results in different assignments of k-mers to partitions based on the number of partitions. This makes inverted indices that have different partition numbers (e.g., constructed by different researchers) difficult to join during the distance matrix computation, reducing the reusability of the indices.

The disclosed methods can employ an approach to partition k-mers based on variable-size k-mer ranges whose size is determined by an approximated k-mer distribution (FIG. 4). To construct the approximated k-mer distribution, called the k-mer histogram, a separate MapReduce job is launched. In the job, k-mers are produced from samples and canonicalized in the same way as in the k-mer counting. However, to reduce the overhead only the first x characters of the k-mers are used in the histogram. This is a trade-off between accuracy and space. If x is a small number, the k-mer histogram will become less accurate but small enough to fit in RAM. Finally, based on the k-mer histogram, the k-mer space is partitioned to have approximately an equal number of k-mers in each partition.

A good value for the prefix length, x, was investigated empirically. FIGS. 5A-5B show changes of the workload imbalance (5A) and the size of histogram (5B) for different values of x (4-8) for k=20 using a real sample in the Pacific Ocean Viromes (POV) dataset (Hurwitz, et al., PloS One, 8:e57355 (2013)). Then, the k-mer space was divided into 100 partitions based on the histograms. Under an assumption that workload of a partition in k-mer counting is proportional to the number of k-mers, k-mers assigned to each partition were counted and compared the number of k-mers to measure the imbalance (5A). When the imbalance threshold was set to 1%, x≥6 showed that all partitions were within the threshold. Also, a k-mer histogram with x=6 required only 32 KB of space (4⁶×8 bytes) to store an array for the k-mer abundances so that it easily fits in memory.

The Spark implementation is similar to the MapReduce implementation except that the samples, histograms, and indices are stored in RDDs rather than files, the Map tasks are replaced by Spark transformations, and the Reduce tasks are replaced with Spark actions.

3. Distance Matrix Computation (Scoring)

To compute the distance matrix, the methods typically utilize a vector space model that produces a vector (Salton, et al., Communications of the ACM, 18(11) 613-620) for each sample. Each dimension of a vector corresponds to a unique k-mer, and its value is the weight given to the corresponding k-mer in the scoring function. The weight is calculated from the k-mer abundance in the inverted indices which uses functions such as logarithmic weighting to dampen an effect of k-mers that are too frequent.

The methods can uses cosine similarity as a distance estimate—the cosine of the angle is proportional to the similarity between the two samples. The cosine is one when the angle is zero (i.e., the vectors are identical except for their magnitude) and less than one otherwise. Therefore, the distance between two samples is 1—similarity.

An advantage of using the cosine similarity is high parallelism. The distance can be efficiently computed in a distributed environment by dividing vectors into ranges of dimensions. The contributions of each range are computed in parallel, and the contributions are merged in a post-processing phase into the distance matrix.

A contribution to the distance between two samples is made at each dimension by multiplying their weights. Therefore, only shared k-mers (i.e., those with non-zero abundance in both samples) contribute to the final score, making efficient determination of shared k-mers important for high performance. A sort-merge join is used to detect shared k-mers. Because the inverted indices have entries already in lexicographic order by k-mer, the sort-merge join can be performed efficiently.

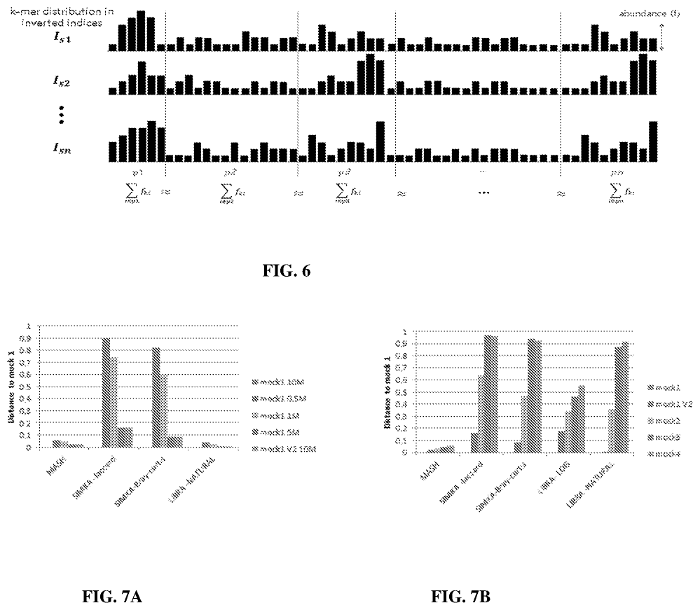

The same histogram-based k-mer range partitioning can be used to split inverted indices. Inverted indices given to the distance matrix computation can be split using k-mer histograms to have approximately equal number of k-mers in each split as described in FIG. 6, under the assumption that the work required to process an input split is proportional to the number of k-mers in the split. A split (p) is assigned for the same k-mer range over all given inverted indices. Also, a split can span multiple chunks in an inverted index depending on k-mer distribution of the sample. Nevertheless, the input-splitting ensures that a k-mer shared between samples is found in the same split because inverted indices have entries in lexicographic order by k-mer.

The disclosed methods can compute the similarity between every pair of samples in the inverted indices. The sort-merge join allows shared k-mers to be found in linear time in the total size of the samples. For those samples that share a particular k-mer, the contributions to the final score are computed between every pair of samples. This requires quadratic time in the number of samples that share the k-mer. While this is potentially a large overhead, in practice it has negligible impact on overall performance because relatively few samples share a k-mer and the pairwise computation consists of multiplication, which finishes quickly.

C. Hadoop

Hadoop (Apache™ Hadoop® available at the hadoop.apache website (2017)), an implementation of the MapReduce algorithm that has been widely adopted for big-data analytics, takes these functions and deploys them across large-scale clusters of machines, allowing these machines to work in concert to process large amounts of data. As a result, programmers do not require specialized training in distributed systems and networking to implement large-scale computations. In addition, Hadoop also provides fault-tolerance. When a Hadoop node fails, Hadoop reassigns the failed node's tasks to another node containing a redundant copy of the data those jobs were processing. This differs from traditional high-performance computing in which schedulers track failed nodes and either restart the failed computations from the most recent checkpoint, or from the beginning if checkpointing was not used. Thus, using Hadoop ensures that computation will complete and data are protected even in the event of hardware failures.

The success of Hadoop has made it ubiquitous, well supported, and well understood. This makes it an appealing platform for scientific computations such as comparative metagenomics as researchers can make use of the existing Hadoop infrastructure and support mechanisms. However, Hadoop was designed for big-data analytics, not for scientific computation, which makes it challenging to use for comparative metagenomic analysis. In particular, some of the techniques Hadoop uses for balancing workloads across tasks are ill-suited for comparative metagenomics.

As discussed above, in some embodiments, input samples are split based on file system data blocks, i.e., a Hadoop Distributed File System (HDFS). HDFS is a technique that defines a file system that can be used to distribute data across multiple computer systems that each store data in a manner that complies with the technique. An HDFS cluster, which can also be referred to as a Hadoop cluster, is a collection of computer systems (sometimes called nodes) storing portions of data in a manner that allows a single operation to be carried out on the portions of data in parallel (e.g., substantially simultaneously). The data of each node is stored using a file system defined by the HDFS technique. The file system is sometimes referred to as HDFS storage. Generally, a file system operating according to HDFS can store any kind of data files. Sometimes a type of file specific to Hadoop, called a sequence file, is used as the file format for data stored in a Hadoop node. A Hadoop cluster could have dozens or hundreds of nodes (or more). In this way, a Hadoop cluster could carry out a single data processing operation across those dozens or hundreds of nodes in parallel, each node operating on a portion of data. Techniques can be used to carry out most or all data processing operations on a Hadoop cluster rather than on a different data processing system that would otherwise perform the operations.

Although a Hadoop node is generally described as a computer system storing a portion of data, a Hadoop node can take other forms. Any arrangement in which a particular portion of data is associated with a particular portion of computer hardware can be a Hadoop node. For example, a single Hadoop node itself could be made up of multiple computer systems, whether they be two or more computer systems operating together to form a node, two processors of a multiprocessor computer system operating together to form a node, or some other arrangement. A single computer system could also act as multiple Hadoop nodes if the single computer system had two distinct file systems operating according to the HDFS technique each with its own portion of data. Further, to say that a node performs a particular action, generally means that the node serves as a platform on which a functional component carries out the described action. For example, a computer program executing on the node may be carrying out the action.

Further, although the Hadoop platform is discussed in this description, other similar techniques that do not carry the Hadoop name, and/or do not use the HDFS data storage format, can be used with the analytical methods described herein. In this way, these same methods can be used with other types of clusters. For example, these methods could be used with another kind of cluster that stores an aggregation of data that can be operated on in parallel by nodes operating in conjunction with one another to carry out a data processing operation on the aggregation of data (e.g., by splitting the aggregation of data into portions operated on by the individual nodes).

One way of processing data in a Hadoop cluster is using a MapReduce programming model. As discussed in more detail elsewhere herein, the disclosed analytical methods typically utilize the MapReduce algorithm. Generally, a MapReduce program includes a Map procedure that performs filtering and sorting (such as sorting university students by first name into queues, one queue for each name) and a Reduce procedure that performs a summary operation (such as counting the number of university students in the respective queues, yielding name frequencies). A user of the system specifies the Map and Reduce procedures, but does not necessarily determine the number of instances (or invocations) of each procedure (i.e., “processes”) or the nodes on which they execute. Rather, a “MapReduce System” (also called “infrastructure,” “framework”) orchestrates by marshaling a set of distributed nodes, running the various tasks (e.g., the Map and Reduce procedures and associated communication) in parallel, managing all communications and data transfers between the various parts of the system, providing for redundancy and failures, and overall management of the whole process. A MapReduce system can schedule execution of instances of Map or Reduce procedures with an awareness of the data location.

Data sources include, but are not limited to, relational databased (sometimes called a Relational Database Management System, or RDBMS), a flat file, a feed of data from a network resource, or any other resource that can provide data in response to a request from the data processing system.

D. Real-Time Analytics

The disclosed methods are also suitable for real-time analysis on platforms such as Spark. Apache Spark is an open-source distributed general-purpose cluster-computing framework, and provides an interface for programming entire clusters with implicit data parallelism and fault tolerance. It features a fast, in-memory data processing engine with expressive development APIs to allow data workers to execute streaming conveniently.

Spark and Hadoop MapReduce are both platforms for data processing. Spark can do it in-memory data processing, while Hadoop MapReduce has to read from and write to a disk. As a result, the speed of processing differs significantly—Spark may be up to 100 times faster. However, the volume of data processed also differs: Hadoop MapReduce is able to work with far larger data sets than Spark. Thus, the user can determine which data processing platform is most suitable for the particular dataset or application at hand.

The disclosed partitioning methodology and associated algorithm(s) enable Spark and other similar platforms to balance the analysis workload across its nodes. This is important because imbalanced workloads increases the overall analysis run time due to nodes with heavy workloads. A heavy workload also requires excessive amounts of memory on the node to store the data associated with the workload. The disclosed methods are also suitable for Spark because the disclosed distance computation methodology and associated algorithm(s) are computationally less intensive than other algorithms, such as Jensen-Shannon. The fast performance and linear scalability of the disclosed method make them particularly suitable for real-time analysis.

Spark utilizes a cluster manager and a distributed storage system. For cluster management, Spark supports standalone (native Spark cluster), Hadoop YARN, or Apache Mesos. For distributed storage, Spark can interface with a wide variety of systems including Alluxio, Hadoop Distributed File System (HDFS), MapR File System (MapR-FS), Cassandra, OpenStack Swift, Amazon S3, Kudu, or a custom solution can be implemented. Through Apache Hadoop YARN, the disclosed methods can exploit Spark's power, derive insights, and enrich the data science workloads within a single, shared dataset in Hadoop.

In some embodiments, the disclosed methods are executed through the particular embodiment of Libra and real-time analysis is carried out using Spark as the data processing platform.

### II. Methods of Use

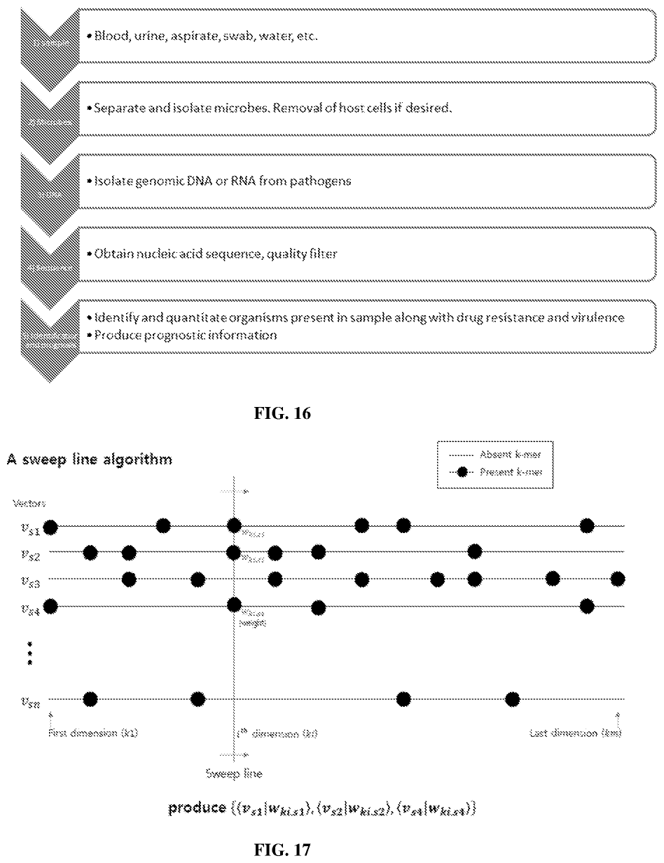

The disclosed comparative genomics approaches can be used across a broad range of analyses. An exemplary workflow is illustrated in FIG. 16.

Disclosed herein are methods of identifying infections, such as methods of identifying bacteria, fungal, viral and/or parasitic infections which utilize whole metagenome sequence analysis to sequence the entire microbiome of sample, for example biological samples, such as the entire wound microbiome. These methods can be used to provide diagnostic and prognostic information about suspected infections, pathogens, microbes, or parasites. In some embodiments, the methods include performing molecular analysis of a biological sample such as a patient wound sample, preparing the data obtained from the molecular analysis, developing diagnostic information about the biological sample (e.g., the wound sample) and/or prognostic information from the sample. Although the methods described herein typically refer to microbial or parasitic infections, which include, without limitation, bacterial, viral, fungal, and parasitic infections, or any combination thereof. It is contemplated that the disclosed methods can be utilized to improve clinical outcomes in many types of infections, including, but not limited to, bacterial, viral, fungal, and parasitic infections. Thus, any reference to microbe or microbial should not be viewed as including or excluding any one or more of bacteria, virus, fungi, or parasite (or bacterial, viral, fungal, and parasitic), or any one or more specific example thereof. Any references to bacteria, virus, fungi, or parasite should also be viewed as contemplating one or more of the other microbes.

In some examples, the disclosed methods are used to diagnose and prognose diabetic foot ulcers (DFUs), sepsis and/or nosocomial infection.

In some examples, the disclosed methods are used to identify biomarkers and/or protein function.

A. Molecular Analysis of Sample

In some examples, molecular analysis of the sample includes obtaining a sample. The sample can be from a biological site, or can be an environmental sample, an industrial sample, a water sample, a soil sample, or an air sample etc. The sample can be a biological sample from a subject, for example a wound sample or a sample from a suspected infection. Biological samples include any sample useful for identifying a microbial or parasitic infection in a subject, including, but not limited to, cells, tissues, and bodily fluids (such as blood or saliva); biopsied or surgically removed tissue, including tissues that are, for example, unfixed, frozen, fixed in formalin and/or embedded in paraffin; tears; skin scrapes; or surface washings. In a particular example, a sample includes cells collected by using a sterile swab or by a surface rinse. In some examples, a sample including nucleic acids is obtained from the subject's wound which is suspected of being infected by microbes by a sterile swab. In some examples, the subject is displaying one or more signs or symptoms of a microbial or parasitic infection, such as inflammation or swelling, redness, presence of pus, increased surface temperature of the wound site, lack-of or delayed wound healing. In some examples, a biological sample is obtained by using the same technique used for obtaining samples for standard culture-based diagnosis in a microbiology laboratory (e.g., a cotton swab).

In some embodiments, molecular analysis of a sample, such a wound sample, includes isolating total DNA from the sample. Total DNA may be isolated by methods disclosed herein as well as those known to those of ordinary skill in the art, including by use of commercially available kits such as the Qiagen EZ1 DSP Virus Kit or DNeasy blood and tissue kit. Regardless of the DNA isolation method used, the resulting DNA sample can be free of contaminants known to inhibit molecular biology procedures, (e.g., hemoglobin, Guanidine Isothiocyanate, phenol) and suspended in an appropriate buffer (e.g., Tris-EDTA buffer). In some examples, DNA is isolated within 24 hours of sample collection and stored at 4° C.

In some examples, the molecular analysis of a sample, such as a wound sample, includes removal of host DNA, as the diagnosis and prognosis may be dependent only on analysis of non-host DNA. Host DNA can be removed from the sample by methods known to those of ordinary skill in the art including those provided herein, including use of commercially available kits (e.g., NEBNext Microbiome DNA Enrichment kit).

In some examples, the molecular analysis of a sample, such as a wound sample includes preparing non-host DNA for sequencing by fragmenting the microbial, or pathogen, or parasitic DNA to the appropriate length for the sequencing platform to be employed. DNA Fragmentation can be performed by methods known to those of skill in the art including enzymatic or physical methods (e.g., Ion Torrent Xpress fragment library kit or sonication on a Corvaris instrument using Adaptive Focused Acoustics technology). The methods disclosed herein are not dependent upon a particular sequencing technology. The user needs to make appropriate DNA fragment size choices for the intended downstream sequencing platform according to manufacturers' protocols. For example, Ion Torrent sequencing technology currently requires targeting a fragment size of up to 400 base pairs. Following fragmentation the microbial DNA is size selected or purified depending on the fragmentation method. The DNA is properly sized (by length in base pairs) for the appropriate technology.

In some embodiments, the molecular analysis of a sample, such as a wound sample, includes sample indexing, adaptor ligation and library normalization. Sample indexing (“barcoding”) allows multiple samples to be run simultaneously taking full advantage of the high-throughput nature of current sequencing platforms. Adapter ligation is sequencing platform specific and standard to manufacturers' protocols. At this step, microbial, or pathogenic, or parasitic DNA fragments have the platform-specific end sequences necessary for sequencing along with index sequences that allow for de-convolution of sequence data by sample. Libraries can be prepared at platform specific concentrations of DNA. Libraries typically require amplification or dilution to achieve the required DNA concentration. The DNA concentration in the library can be determined by quantitative real-time PCR using platform specific manufacturer protocols or fluorescence-based measurement using an instrument such as the ThermoFisher Qubit. The sequencing library represents the fragments of DNA that make up the genome of the microbes present in the patient sample. These are the molecules whose sequence is determined to generate reads that can be used for k-mer generation and subsequent analyses.

In some embodiments, the molecular analysis of a sample, such as a wound sample, includes performing whole metagenome sequence analysis to sequence the entire wound microbiome of the sample provided. For example, nucleotide sequences of individual molecules are determined in a platform specific manner to produce raw data. Raw data is converted to nucleotide sequencing information for each molecule in the library in a platform-specific manner. The resulting products are whole metagenome “reads.” At this point, the DNA of the microbes has been converted to binary computer information represented in a “BAM file” that can be processed to determine information about the clinically important sample composition. BAM files are sequencing platform independent and ready for bioinformatics analysis. Additional file types may include FASTA and FASTQ file formats or other manufacturer-specific formats what can be converted to BAM, FASTQ, or FASTA format.

In some embodiments, sequence data may be transferred in real time from the instrument generating sequence data as soon as the sequence data has reached a sufficient size in total base-pairs for analysis.

B. Data Preparation

In some embodiments, data preparation includes performing sequence quality control. In some embodiments, the resulting BAM or FASTQ file of reads from the molecular analysis is subjected to quality trimming, length filtering, sequencing adapter removal and binning of reads by molecular barcode. In particular, the reads that represent the DNA sequence can be quality controlled to remove the platform specific adapters, clonal reads due to PCR amplification, and platform-specific sequence errors and filtered to achieve an acceptable error rate.

In some embodiments, following quality control and trimming, reads that are less than two standard deviations from the mean length are discarded. Due to the high throughput of next generation sequencing, samples can be multiplexed within a single run. The indexing is achieved by the addition of a molecular bar-code consisting of sample specific sequence added to the sequencing adapters during library preparation. Following quality control, sequences are de-convoluted to create sample specific reads by analyzing the molecular barcode at the start of each reads and binning it accordingly. At this stage, reads still contain the molecular barcodes and sequence adapters used to generate them. This sequence does not contain diagnostic or prognostic information and can be removed before diagnosis or clinical prognosis analysis. Adapters can be removed using methods known to those of skill in the art that have been standardized to account for read errors, chimeric reads, reverse complement reads, and fragmented adapters. The resulting quality controlled sequence reads with acceptable and known error rate (e.g., phred quality score of 20 or higher at each base in the read), are the appropriate length, and contain only biologically derived sequence. The end result of the quality filtering steps are reads representing biological information free of technical errors from the sequencing process.

In some examples, data preparation includes removal of host sequence reads. The physical removal of patient-derived DNA during sequencing library preparation is not 100% efficient. Therefore some of the reads will be derived from patient sequence and are irrelevant with respect to diagnosis or prognosis. In addition, the patient-derived reads could lead to privacy issues through inadvertent analysis of the genetic content of the patient's genome. Therefore, in some embodiments, the final step of quality control is in silico removal of reads derived from host DNA. Following removal of host sequence reads, microbe-specific sequence reads are provided. At this stage, remaining reads are high quality, appropriate length, and of microbial, pathogenic, or parasitic origin. This represents the raw starting material for computational analyses of clinical importance (e.g. generating diagnostic and/or prognostic information).

Data preparation can include deconstructing reads into k-mers of approximately 20 bases. The k-mer size can range from 20-100 bases, and can be set by, for example, examining the uniqueness ratio in the dataset (Kurtz et al., BMC Genomics, 9:517 (2008), which is hereby incorporated by reference in its entirety) the k-mer value is chosen by finding the inflection point where the k-mer hits move from “random” to representative of the sequence content. In the case of diagnosis, k-mers can be derived from the genomic sequence of known microbes. In the case of prognosis, the k-mers can be derived from sequencing similar patient samples for which the clinical outcome is known (e.g., healed versus chronic wound).

Deconstructing the sequencing reads (typically of 100-600 base pairs in length) into k-mers of approximately 20 base pairs in length (ranging between 20-100 bases) avoids the complex problems that arise from attempting to assemble genomes, problems that are exacerbated by the likely presence of multiple independent genomes in poly-microbial samples and the low coverage anticipated for each genome. In the case of approaches that do not attempt genome assembly, but use read to read pairwise comparison (e.g., BLAST or clustering methods such as cdhit or usearch), there is no computationally efficient way to solve the problem, leading to even simple cases becoming intractable given finite computational resources.

The k-mers can be utilized in the disclosed methods of genetic sequence analysis.

C. Diagnostics

In some embodiments, the disclosed methods are utilized to diagnose a subject with, for example, an infection. Patient diagnosis can include utilizing information obtained by comparing the k-mers derived from sample reads to k-mers of known microbial, pathogenic, or parasitic reference sequences held in a pre-existing database. The analysis can be a pairwise all-versus-all problem made computationally possible by the use of short k-mers and the disclosed analytical strategies. Again, in the case of diagnosis, k-mers derived from reads of the biological sample can be compared to k-mers derived from reads of reference sample(s) of known identity or other prior samples from, for example, patients with similar clinical conditions. Reference sequences for comparison to the sample may be derived from publicly available data, prior samples, or sequence generated by the patent holders or their agents; and may include whole or partial genomic sequences along with sequences of specific chromosomes, genes, gene fragments, plasmids, or pathogenicity islands. In the case of prognosis (as discussed in more detail below), biological sample k-mers can be compared to k-mers derived from reads of other patient samples of known clinical outcome. The samples can be of known or unknown identity and each sample analyzed, along with relevant metadata, may become part of the reference for the next sample analyzed.

Diagnosing a subject can include summarizing the results of comparative genomics analysis into a clinical report. For example, the results can be summarized into a simple table of species found in the sample along with their relative proportion in the sample with additional flags indicating the presence of antibiotic resistance genes.

Providing diagnostic information on a subject can include providing the results, findings, identifications, relative abundance estimates, predictions and/or treatment recommendations for the subject. For example, the results, findings, identifications, predictions and/or treatment recommendations can be recorded and communicated to technicians, physicians and/or patients or clients. In certain embodiments, computers will be used to communicate such information to interested parties, such as, clients, patients and/or the attending physicians.

In some embodiments, once a subject's non-host sequences are identified, an indication of that identity can be displayed and/or conveyed to a clinician, caregiver or a non-clinical provider, including the client/subject. For example, the results of the test are provided to a user (such as a clinician or other health care worker, laboratory personnel, or patient) in a perceivable output that provides information about the results of the method. The output can be, for example, a paper output (for example, a written or printed output), a display on a screen, a graphical output (for example, a graph, chart, or other diagram), or an audible output.

In some embodiments, the output is a numerical value, such as an amount of a particular set of sequence in the sample as compared to a control. In additional examples, the output is a graphical representation, for example, a graph that indicates the value (such as amount or relative amount) of the particular microbes in the sample from the subject on a standard curve. In a particular example, the output (such as a graphical output) shows or provides a cut-off value or level that indicates the presence of a microbes or parasites that could cause an infection. In some examples, the output is communicated to the user, for example by providing an output via physical, audible, or electronic means (for example by mail, telephone, facsimile transmission, email, or communication to an electronic medical record).

The output can provide quantitative information (for example, an amount of a molecule in a test sample compared to a control sample or value) or can provide qualitative information (moderate to severe microbial infection caused by a particular microbe or parasite indicated). In additional examples, the output can provide qualitative information regarding the relative amount of a particular microbe(s) in the sample, such as identifying presence of an increase relative to a control, a decrease relative to a control, or no change relative to a control.

In some embodiments, the output is accompanied by guidelines for interpreting the data, for example, numerical or other limits that indicate the presence or absence of a particular microbial or parasitic disorder/condition. The indicia in the output can, for example, include normal or abnormal ranges or a cutoff, which the recipient of the output may then use to interpret the results, for example, to arrive at a diagnosis, prognosis, susceptibility towards or treatment plan. In some examples, the findings are provided in a single page report (e.g., PDF file) for the healthcare provider to use in clinical decision making.

Based on the findings, the therapy or protocol administered to a subject can be started, modified not started or restarted (in the case of monitoring for a reoccurrence of a particular condition/disorder). Recommendations of what treatment to provide can be provided either in verbal or written communication. The recommendations can be provided to the individual via a computer or in written format and accompany the diagnostic report. For example, a subject may request their report and suggested treatment protocols be provided to them via electronic means, such as by email.

The report can include determination of other clinical or non-clinical information.

In some embodiments, the communication containing the diagnostic information and/or treatment recommendations or protocols based on the results, may be generated and delivered automatically to the subject using a combination of computer hardware and software which will be familiar to artisans skilled in telecommunications. One example of a healthcare-oriented communications system is described in U.S. Pat. No. 6,283,761; however, the present disclosure is not limited to methods which utilize this particular communications system. In certain embodiments of the methods of the disclosure, all or some of the method steps, including the assaying of samples, performing the comparisons, and/or communicating of assay results, diagnoses or recommendations, may be carried out together or separately and in the same or in diverse (e.g., foreign) locations.

In some embodiments, the treatment, dose or dosing regimen is modified based on the information obtained using the disclosed methods.

A subject can be monitored while undergoing treatment using the methods described herein in order to assess the efficacy of the treatment or protocol. In this manner, the length of time or the amount of treatment given to the subject can be modified based on the results obtained using the methods disclosed herein. The subject can also be monitored after the treatment using the methods described herein to monitor for relapse and thus, the effectiveness of the given treatment. In this manner, whether to resume treatment can be decided based on the results obtained using the methods disclosed herein. In some embodiments, this monitoring is performed by a clinical healthcare provider. This monitoring can be performed by a non-clinical provider and can include self-monitoring or monitoring by a weight consultant.

D. Prognosis

Methods of prognosis are also provided. Clinical prognosis typically includes comparing k-mers from biological sample derived reads to, for example, prior samples (e.g., clinical samples) for which outcome is known. In some embodiments, the results of the comparative genomics analysis are summarized into a sample distance matrix.

Clinical outcome may be determined by analyzing statistically the probabilistic distance a patient sample is from other samples of known outcomes and reporting such as a risk (e.g., risk the patient's wound will be chronic). For example, a deliverable diagnostic report (e.g., PDF file) may be generated for the physician to use in clinical decision making indicating whether a patient sample belongs to a particular prior grouping (healed versus chronic wound). Distances between samples for statistical analyses and visualization can be carried out using clustering methods including, but not limited to, partitioning methods, hierarchical clustering, density-based methods, model-based clustering methods, grid-based methods, and soft-clustering. Typical clustering methods include: k-means clustering, bayesian network analyses, mean shift clustering, density-based spatial clustering of applications with noise (DBSCAN), expectation maximization using Gaussian Mixture Models (GMM), Agglomerative Hierarchical Clustering. K-mers may also be normalized for specific clinical applications using techniques such as Boolean weighting, logarithmic, and natural log for enhanced visualization of results in clinical reports. Additional formats may be utilized to provide the results including those discussed herein as well as those known to those of ordinary skill in the art.

The sequence reads that drive prognosis and diagnosis are also extractable from the total data. These reads can be translated in silico into putative protein sequence and analyzed against protein motif databases to identify protein functions that correlate significantly with clinical information (e.g., protease or beta-lactamase activity correlating with tissue invasion or antibiotic resistance). In addition, the sequence reads could provide new biomarkers for the development of rapid diagnostic assays. Thus the disclosed methods can be used to identify protein function as well as new biomarkers.

E. Providing a Treatment/Protocol to a Subject

In some embodiments, the methods further include providing an appropriate therapy or protocol for the subject after reviewing the diagnostic and/or prognostic results. For example, a subject diagnosed with a particular microbial or parasitic infection can be provided a particular therapy. In some examples, the therapy includes administering an agent to alter one or more signs or symptoms associated with the identified microbial or parasitic disorder/condition. In some examples, the therapy may be altered to adapt to the emergence or change in abundance of new microbes, pathogens, or parasites. The treatment/protocol can be performed multiple times for optimal results. In one embodiment, the treatment is performed twice a day. In another embodiment, the treatment is performed daily. In other embodiments, the recommendation/treatment is performed weekly. In another embodiment, the treatment is performed monthly. In another embodiment, the treatment is performed at least once every one to two days. In another embodiment, the treatment is performed at least once every one to two weeks.

The desired treatments or protocols can be administered via any means known to one of skill in the art, including, but not limited to, oral, topical, or systemic administration. In some examples, a composition is administered to the subject orally, such as in a capsule or tablet. One or more compositions can be administered via multiple routes at the same or different time period depending upon the disorders/conditions being treated. The percentage of improvement can be, for example, at least about a 5%, such as at least about 10%, at least a 15%, at least a 20%, at least about 30%, at least about 40%, at least about 50%, at least about 60%, at least about 70%, at least about 80%, at least about 90% or at least about 100% change compared to the baseline score prior to treatment with one or more microbial altering/controlling agents. The improvement can be measured by both subjective and objective methods, and can be quantified using a subjective scoring or a panel scoring, amongst other methods.

F. Types of Organisms Detected in a Sample

The disclosed methods can be used to identify the presence of organisms and infections thereof, such as bacteria, fungal, viral and/or parasitic. In one example, one or more of the following types of organisms can be detected by the present method: Abiotrophia, Acanthamoeba, Acetobacteraceae, Achromobacter, Acidaminococcus, Acidithiobacillus, Acidocella, Acidovorax, Acinetobacter, Acremonium, Actinobacillus, Actinobaculum, Actinomadura, Actinomyces, Adenovirus, Aerococcus, Aeromonas, Aeropyrum, Aggregatibacter, Agrobacterium, Akkermansia, Alcaligenes, Alistipes, Alphacoronavirus, Alternaria, Alteromonas, Anabaena, Anaerobiospirillum, Anaerococcus, Anaeroglobus, Anaerostipes, Anaplasma, Anoxybacillus, Aquabacterium, Arachnia, Aranicola, Arcanobacterium, Arcobacter, Arthrobacter, Arthroderma, Arthrospira, Ascaris, Aspergillus, Astrovirus, Atopobium, Bacillus, Bacteroides, Bacteroidetes, Bartonella, Beauveria, Betacoronavirus, Bifidobacterium, Bilophila, Bipolaris, Blastochloris, Blastococcus, Blastocystis, Blastomyces, Blastoschizomyces, Blautia, Bordetella, Borrelia, Brachymonas, Brachyspira, Bradyrhizobium, Branhamella, Brevibacillus, Brevibacterium, Brevundimonas, Brucella, Buchnera, Bulleidia, Burkholderia, Burkholderiales, Buttiauxella, butyrate-producing organism, Butyrivibrio, Calicivirus, Campylobacter, Candida, Candidatus, Capnocytophaga, Carbolfuchsin, Cardiobacterium, Carnobacterium, Catenibacterium, Caulobacter, Cedecea, Cefuroxime, Cellulosimicrobium, Centipeda, Cephalosporins, Cephalosporium, Chaetomium, Chaetothyriales, Chilomastix, Chlamydia, Chlamydophila, Chromobacterium, Chryseobacterium, Chrysosporium, Citrobacter, Cladosporium, Clarithromycin, Clindamycin, Cloacibacterium, Clonorchis, Clostridiales, Clostridium, Coccidioides, Collinsella, Comamonas, Conidiobolus, Coprobacillus, Coprococcus, Corynebacteria, Corynebacterium, Coxiella, Cryptobacterium, Cryptococcus, Cryptosporidium, Cunninghamella, Curvularia, Cyanobacteria, Cyclospora, Cylindrospermopsis, Cytomegalovirus, Dactylaria, Davidiella, Delftia, Deltacoronavirus, Dermabacter, Desmospora, Desulfitobacterium, Desulfomicrobium, Desulfovibrio, Dialister, Didymella, Dientamoeba, Diphyllobothrium, Dolosigranulum, Dorea, Dreschlera, Eboli, Echinococcus, Edwardsiella, Eggerthella, Ehrlichia, Eikenella, Empedobacter, Enhydrobacter, Entamoeba, Enterobacter, Enterobacteriaceae, Enterobius, Enterococci, Enterococcus, Enterovirus, Epicoccum, Epidermophyton, Eremococcus, Erwinia, Erysipelothrix, Erysipelotrichaceae, Erythrobacter, extended spectrum beta-lactamase(ESBL), Escherichia, Eubacterium, Ewingella, Excerohilum, Exiguobacterium, Exoantigen, Exophiala, Facklamia, Faecalibacterium, Filifactor, Finegoldia, Flavobacterium, Flavonifractor, Fonsecaea, Francisella, Frankia, Fusarium, Fusobacterium, Gallicola, Gammacoronavirus, Gardnerella, Gemella, Geobacillus, Geotrichum, Giardia, Giemsa, Gliocladium, Gordonia, Gordonibacter, Granulicatella, Haemophilus, Hafnia, Haloarcula, Halobacterium, Halosimplex, Hansenula, Helcococcus, Helicobacter, Helminthosporium, Hemadsorbing, Herpes, Histoplasma, Holdemania, Hymenolepis, Hyphomicrobium, Iodamoeba, Isospora, Janibacter, Janthinobacterium, Jeotgalicoccus, Johnsonella, Kingella, Klebsiella, Kluyvera, Kocuria, Koserella, Lachnospiraceae, Lactobacillus, Lactococcus, Lautropia, Leclercia, Legionella, Leifsonia, Leminorella, Leptospira, Leptotrichia, Leuconostoc, Listeria, Listonella, Lyngbya, Lysinibacillus, Malassezia, Malbranchea, Mannheimia, Megamonas, Megasphaera, Mesorhizobium, Methanobacterium, Methanobrevibacter, Methanosaeta, Methanosarcina, Methanothermobacter, Methylobacterium, Microbacterium, Micrococcus, Microcoleus, Microcystis, Microsporidia, Microsporum, Mobiluncus, Mogibacterium, Mollicutes, Moraxella, Morganella, Mycelia, Mycetocola, Mycobacterium, Mycoplasma, Myroides, Neisseria, Neorickettsia, Nigrospora, Nocardia, Nodularia, Nostoc, Oceanobacillus, Ochrobactrum, Odoribacter, Oenococcus, Oerskovia, Oligella, Olsenella, Oribacterium, Ornithobacterium, Oscillatoria, Oxalobacter, Paecilomyces, Paenibacillus, Pantoea, Parabacteroides, Paracoccus, Paraprevotella, Parascardovia, Parasutterella, Parvimonas, Pasteurella, Pediculus, Pediococcus, Penicillium, Peniophora, Peptococci, Peptococcus, Peptoniphilus, Peptostreptococcus, Petrobacter, Phaeoacremonium, Phaeoannellomyces, Phascolarctobacterium, Phialemonium, Phialophora, Photobacterium, Photorhabdus, Phyllobacterium, Pichia, Picornavirus, Pirellula, Piscirickettsia, Planktothrix, Planomicrobium, Plasmodium, Plesiomonas, Pneumocystis, Poliovirus, Porphyromonas, Prevotella, Propionibacterium, Proteus, Prototheca, Providencia, Pseudallescheria, Pseudomonas, Pseudoramibacter, Pseudoxanthomonas, Rahnella, Ralstonia, Raoultella, Rathayibacter, Rhinocladiella, Rhinosporidium, Rhinovirus, Rhizobium, Rhizomucor, Rhizopus, Rhodanobacter, Rhodococcus, Rhodopirellula, Rhodopseudomonas, Rhodotorula, Riemerella, Roseburia, Roseomonas, Rotavirus, Rothia, Ruminococcaceae, Ruminococcus, Saccharomyces, Salmonella, Sarcoptes, Scardovia, Scedosporium, Schistosoma, Schizophyllum, Schlegelella, Scopulariopsis, Scytalidium, Segniliparus, Selenomonas, Sepedonium, Serratia, Shewanella, Shigella, Simonsiella, Sistotrema, Slackia, Sneathia, Solobacterium, Sphingobacterium, Sphingobium, Sphingomonas, Spirochaeta, Spirochaetaceae, Spirochetes, Spirosoma, Sporobolomyces, Sporothrix, Stachybotrys, Staphylococcus, Stemphylium, Stenotrophomonas, Stenoxybacter, Streptococcus, Streptomyces, Strongyloides, Succinatimonas, Succinivibrio, Sutterella, Syncephalastrum, Synechococcus, Synergistetes, Taenia, Tannerella, Tatumella, Tepidimonas, Tetragenococcus, Tissierella, Treponema, Trichinella, Trichoderma, Trichomonads, Trichomonas, Trichophyton Trichosporon, Trichothecium, Trichuris, Tropheryma, Trypanosoma, Turicibacter, Udeniomyces, Ulocladium, Ureaplasma, Ureibacillus, Ustilago, Vagococcus, Varicella, Variovorax, Veillonella, Verticillium, Vibrio, Virgibacillus, Viridans, Vulcanisaeta, Wangiella, Wautersia, Weeksella, Weissella, Wolbachia, Wolinella, Xanthomonas, Xylohypha, Yersinia, Yokenella, Zoogloea, or Zygomycete.

In some examples, one or more pathogenic bacteria are detected with the disclosed method. Examples of pathogenic bacteria which could be detected with the disclosed methods include without limitation any one or more of (or any combination of: Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginals, Alcaligenes xylosoxidans, Acinetobacter baumanii, Actinobacillus actinomycetemcomitans, Bacillus sp. (such as Bacillus anthracia, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtherias, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio fumisii), Yersinia sp. (such as Yersinia enterocolitica, Yersinia pestis, and Yersinia pseudotuberculosis) and Xanthomonas maltophilia among others.

In some examples, one or more pathogenic fungi are detected with the disclosed method. Examples of pathogenic fungi which could be detected with the disclosed methods include without limitation any one or more of (or any combination of) Trichophyton rubrum, T mentagrophytes, Epidermophyton floccosum, Microsporum canis, Pityrosporum orbiculare (Malassezia furfur), Candida sp. (such as Candida albicans), Aspergillus sp. (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), Histoplasma sp. (such as Histoplasma capsulatum), Pneumocystis sp. (such as Pneumocystis jirovecii), and Stachybotrys (such as Stachybotrys chartarum) among others.

In some examples, one or more viruses are detected with the disclosed method. Examples of viruses which could be detected with the disclosed methods include without limitation any one or more of (or any combination of) Arenaviruses (such as Guanarito virus, Lassa virus, Junin virus, Machupo virus and Sabia), Arteriviruses, Roniviruses, Astroviruses, Bunyaviruses (such as Crimean-Congo hemorrhagic fever virus and Hantavirus), Barnaviruses, Birnaviruses, Bornaviruses (such as Borna disease virus), Bromoviruses, Caliciviruses, Chrysoviruses, Coronaviruses (such as Coronavirus and SARS), Cystoviruses, Closteroviruses, Comoviruses, Dicistroviruses, Flaviruses (such as Yellow fever virus, West Nile virus, Hepatitis C virus, and Dengue fever virus), Filoviruses (such as Ebola virus and Marburg virus), Flexiviruses, Hepeviruses (such as Hepatitis E virus), human adenoviruses (such as human adenovirus A-F), human astroviruses, human BK polyomaviruses, human bocaviruses, human coronavirus (such as a human coronavirus HKU1, NL63, and OC43), human enteroviruses (such as human enterovirus A-D), human erythrovirus V9, human foamy viruses, human herpesviruses (such as human herpesvirus 1 (herpes simplex virus type 1), human herpesvirus 2 (herpes simplex virus type 2), human herpesvirus 3 (Varicella zoster virus), human herpesvirus 4 type 1 (Epstein-Barr virus type 1), human herpesvirus 4 type 2 (Epstein-Barr virus type 2), human herpesvirus 5 strain AD169, human herpesvirus 5 strain Merlin Strain, human herpesvirus 6A, human herpesvirus 6B, human herpesvirus 7, human herpesvirus 8 type M, human herpesvirus 8 type P and Human Cyotmegalovirus), human immunodeficiency viruses (HIV) (such as HIV 1 and HIV 2), human metapneumoviruses, human papillomaviruses (such as human papillomavirus-1, human papillomavirus-18, human papillomavirus-2, human papillomavirus-54, human papillomavirus-61, human papillomavirus-cand90, human papillomavirus RTRX7, human papillomavirus type 10, human papillomavirus type 101, human papillomavirus type 103, human papillomavirus type 107, human papillomavirus type 16, human papillomavirus type 24, human papillomavirus type 26, human papillomavirus type 32, human papillomavirus type 34, human papillomavirus type 4, human papillomavirus type 41, human papillomavirus type 48, human papillomavirus type 49, human papillomavirus type 5, human papillomavirus type 50, human papillomavirus type 53, human papillomavirus type 60, human papillomavirus type 63, human papillomavirus type 6b, human papillomavirus type 7, human papillomavirus type 71, human papillomavirus type 9, human papillomavirus type 92, and human papillomavirus type 96), human parainfluenza viruses (such as human parainfluenza virus 1-3), human parechoviruses, human parvoviruses (such as human parvovirus 4 and human parvovirus B19), human respiratory syncytial viruses, human rhinoviruses (such as human rhinovirus A and human rhinovirus B), human spumaretroviruses, human T-lymphotropic viruses (such as human T-lymphotropic virus 1 and human T-lymphotropic virus 2), Human polyoma viruses, Hypoviruses, Leviviruses, Luteoviruses, Lymphocytic choriomeningitis viruses (LCM), Marnaviruses, Narnaviruses, Nidovirales, Nodaviruses, Orthomyxoviruses (such as Influenza viruses), Partitiviruses, Paramyxoviruses (such as Measles virus and Mumps virus), Picornaviruses (such as Poliovirus, the common cold virus, and Hepatitis A virus), Potyviruses, Poxviruses (such as Variola and Cowpox), Sequiviruses, Reoviruses (such as Rotavirus), Rhabdoviruses (such as Rabies virus), Rhabdoviruses (such as Vesicular stomatitis virus, Tetraviruses, Togaviruses (such as Rubella virus and Ross River virus), Tombusviruses, Totiviruses, Tymoviruses, Noroviruses, bovine herpesviruses including Bovine Herpesvirus (BHV) and malignant catarrhal fever virus (MCFV), among others.

Exemplary parasites that can be identified with the disclosed methods herein include, but are not limited to, Malaria (Plasmodium falciparum, P. vivax, P. malariae), Schistosomes, Trypanosomes, Leishmania, Filarial nematodes, Trichomoniasis, Sarcosporidiasis, Taenia (T. saginata, T. solium), Leishmania, Toxoplasma gondii, Trichinelosis (Trichinella spiralis) and/or Coccidiosis (Eimeria species).

In some examples, a diabetic foot ulcer is identified by detecting an organism in one or more of the following genus: Acinetobacter, Corynebacterium, Enterococcus, and/or Pseudomonas.

In some examples, a diabetic foot ulcer is identified by detecting one or more of the organisms: Acinetobacter baumannii-calcoaceticus, Corynebacterium auri, Corynebacterium ssp., Corynebacterium striatum, Corynebacterium striatum/amycolatum, Enterococcus faecalis, and/or Pseudomonas aeruginosa.

In one example, a diabetic foot ulcer is identified by detecting one or more of the following organisms: Acinetobacter baumannii-calcoaceticus Staphylococcus aureus, Acinetobacter baumannii-calcoaceticus Staphylococcus epidermidis, Corynebacterium auris Staphylococcus haemolyticus, Corynebacterium spp. Staphylococcus aureus, Corynebacterium spp. Staphylococcus spp., Corynebacterium striatum Staphylococcus aureus, Corynebacterium striatum/amycolatum Staphylococcus aureus, Corynebacterium striatum/amycolatum Staphylococcus caprae, Corynebacterium striatum/amycolatum Staphylococcus haemolyticus, Enterococcus faecalis Corynebacterium macginleyi, Enterococcus faecalis Corynebacterium striatum, Enterococcus faecalis Staphylococcus aureus, Enterococcus faecalis Staphylococcus capitis, Enterococcus faecalis Staphylococcus epidermidis, Enterococcus faecalis Staphylococcus hominis, Enterococcus faecalis Staphylococcus sp., Pseudomonas aeruginosa Enterococcus faecalis and/or Pseudomonas aeruginosa Enterococcus faecium.

### III. Exemplary Computing Environment

One or more of the above-described techniques may be implemented in or involve one or more computer systems. FIG. 15 illustrates a generalized example of a computing environment 600. The computing environment 600 is not intended to indicate any limitation as to scope of use or functionality of described embodiments.

With reference to FIG. 6, the computing environment 600 includes at least one processing unit 610 and memory 620. In FIG. 6, this basic configuration 630 is included within a dashed line. The processing unit 610 executes computer-executable instructions and may be a real or a virtual processor. In a multi-processing system, multiple processing units execute computer-executable instructions to increase processing power. The memory 620 may be volatile memory (e.g., registers, cache, RAM), non-volatile memory (e.g., ROM, EEPROM, flash memory, etc.), or some combination of the two. In some embodiments, the memory 620 stores software 680 implementing described techniques.

A computing environment may have additional features. For example, the computing environment 600 includes storage 640, one or more input devices 650, one or more output devices 660, and one or more communication connections 670. An interconnection mechanism (not shown) such as a bus, controller, or network interconnects the components of the computing environment 600. Typically, operating system software (not shown) provides an operating environment for other software executing in the computing environment 600, and coordinates activities of the components of the computing environment 600.

The storage 640 may be removable or non-removable, and includes magnetic disks, magnetic tapes or cassettes, CD-ROMs, CD-RWs, DVDs, or any other medium which may be used to store information and which may be accessed within the computing environment 600. In some embodiments, the storage 640 stores instructions for the software 680.

The input device(s) 650 may be a touch input device such as a keyboard, mouse, pen, trackball, touch screen, or game controller, a voice input device, a scanning device, a digital camera, or another device that provides input to the computing environment 600. The output device(s) 660 may be a display, printer, speaker, or another device that provides output from the computing environment 600.

The communication connection(s) 670 enable communication over a communication medium to another computing entity. The communication medium conveys information such as computer-executable instructions, audio or video information, or other data in a modulated data signal. A modulated data signal is a signal that has one or more of its characteristics set or changed in such a manner as to encode information in the signal. By way of example, and not limitation, communication media include wired or wireless techniques implemented with an electrical, optical, RF, infrared, acoustic, or other carrier.

Implementations may be described in the general context of computer-readable media. Computer-readable media are any available media that may be accessed within a computing environment. By way of example, and not limitation, within the computing environment 600, computer-readable media include memory 620, storage 640, communication media, and combinations of any of the above.

Typically, the disclosed analytical methods are deployed via a Hadoop cluster. Hadoop clusters typically run Hadoop's open source distributed processing software on low-cost commodity computers. Typically one machine in the cluster is designated as the NameNode and another machine the as JobTracker; these computer are referred to as the masters. The rest of the computers in the cluster act as both DataNode and TaskTracker; these computers are referred to as slaves. Hadoop clusters can be referred to as “shared nothing” systems because the network that connects them is the only thing that is shared between nodes.

Hadoop is an increasingly attractive platform for performing large-scale sequence analysis because it provides a distributed file system and the MapReduce framework for analyzing massive amounts of data. Hadoop clusters are composed of commodity servers so that the processing power increases as more computing resources are added. If a cluster's processing power is overwhelmed by growing volumes of data, additional cluster nodes can be added to increase throughput. Hadoop also offers a high-level programming abstraction based on MapReduce that greatly simplifies the implementation of new analytical tools. Programmers do not require specialized training in distributed systems and networking to implement distributed programs using Hadoop.

These benefits have led to new analytic tools based on Hadoop, making Hadoop a de facto standard in large-scale data analysis. The amount of sequence data is increasing due to the development of new high-throughput sequencing technologies. The increased data scale often makes existing analytic tools unavailable due to their lack of scalability. Therefore, Hadoop is emerging as a possible solution to performing massive bioinformatics analyses.

The disclosed methods provide a scalable algorithm, and an exemplary embodiment, Libra, that is capable of performing all-vs-all sequence analysis using MapReduce on the Apache Hadoop platform. It can be ported to any MapReduce cluster (e.g., Wrangler at TACC, Amazon EMR or private Hadoop clusters).

The disclosed methods also provide a scalable algorithm, and an exemplary embodiment, Libra, that is capable of performing all-vs-all sequence analysis using Spark optionally executed from the Apache Hadoop platform. This approach facilitates real-time data processing and analytics.

### IV. Non-Transitory Computer-Readable Media

Any of the computer-readable media herein can be non-transitory (e.g., volatile or non-volatile memory, magnetic storage, optical storage, or the like).

### V. Storing in Computer-Readable Media

Any of the storing actions described herein can be implemented by storing in one or more computer-readable media (e.g., computer-readable storage media or other tangible media).

Any of the things described as stored can be stored in one or more computer-readable media (e.g., computer-readable storage media or other tangible media).

### VI. Methods in Computer-Readable Media

Any of the methods described herein can be implemented by computer-executable instructions in (e.g., encoded on) one or more computer-readable media (e.g., computer-readable storage media or other tangible media). Such instructions can cause a computer to perform the method. The technologies described herein can be implemented in a variety of programming languages.

### VII. Methods in Computer-Readable Storage Devices

Any of the methods described herein can be implemented by computer-executable instructions stored in one or more computer-readable storage devices (e.g., memory, magnetic storage, optical storage, or the like). Such instructions can cause a computer to perform the method.

### EXAMPLES

### Example 1: Libra Computational Strategy

### Materials and Methods

Experiment Environment Description

Hadoop Cluster Configuration.

The Libra experiments described below were performed on a Hadoop cluster consisting of 10 physical nodes (9 MapReduce worker nodes). Each node contains 12 CPUs and 128 GB of RAM, and is configured to run a maximum of 7 YARN containers simultaneously with 10 GB of RAM per container. The remaining system resources are reserved for the operating system and other Hadoop services such as Hive or Hbase.

Libra Algorithm Description.

K-Mer Size.

There are several considerations for choosing the k-mer size k. Larger values of k result in fewer matches due to sequencing errors and fragmentary metagenomic data. However, smaller values of k give less information about the sequence similarities. In Libra, k is a configurable parameter chosen by the user. For the analysis in experiments described below, k was set to equal 20. This value has been determined in the literature to be at the inflection point where the k-mer matches move from random to representative of the read content, and is generally resilient to sequencing error and variation (Hurwitz, et al., PNAS, 111:10714-10719 (2014); Kurtz, et al., BMC Genomics, 9:517 (2008)).

Scoring.

Libra uses a vector space model to compute the distance between two NGS datasets. In this model each sample is represented by a vector, each dimension of which corresponds to a unique k-mer. Each component of a vector indicates the weight given to the corresponding k-mer in the scoring function. For example, using the frequency (the raw count) of a k-mer as its weight and using 4-mers, the vector <2,4,0, . . . > indicates that a k-mer ‘aaaa’ has a weight of two and a k-mer ‘aaac’ has a weight of four in the sample, etc.

The distance between two samples can now be measured by comparing their vectors; specifically the angle between the two vectors is an estimate of their genetic distance. The larger the angle, the larger the distance. Libra uses cosine similarity as a distance estimate—the cosine of the angle is proportional to the similarity between the two samples. The cosine is one when the angle is zero (i.e. the vectors are identical except for their magnitude) and less than one otherwise.

The cosine of the angle does not depend on the magnitude (length) of the vectors. This is advantageous in comparing samples with different sizes of samples (or sequencing depth). For example, if there are two samples with the same composition of k-mers but one has k-mers with double the frequency than the other, their vectors will have same angles so that their cosine similarity will one.

One of the benefits of Libra is that it can efficiently perform distance comparisons between all pairs of samples in the input dataset. In general, this would require n×(n−1)/2n×(n−1)/2 comparisons for n samples, which becomes prohibitively expensive as n gets large. One possibility is to perform each pairwise comparison in parallel on a cluster of computers, but even that becomes expensive as each sample is involved in n comparisons and therefore may have to be processed by up to n different computers. Importantly, Libra performs the computations in such a way as to greatly reduce the computational cost.

Libra constructs a vector vₛvₛ for each sample s from the weight of each k-mer k in the sample (w_{k,s}w_{k,s}). Each dimension in the vector corresponds to the weight of the corresponding k-mer:

vₛ=(w_{k1,s},w_{k2,s},w_{k3,s}, . . . ,w_{kn,s})

The weight of a k-mer in a sample (wₖₛwₖₛ) can be derived from frequency of the k-mer (fₖₛfₖₛ) in several ways. The simplest way is using the raw frequency of the k-mer (wₖₛ=fₖₛwₖₛ=fₖₛ), called Natural Weighting. Another way is using Logarithmic Weighting (w_{kz}=1+log(f_{kz}) wₖₛ=1+log(fₖₛ) to not give too much weight to highly abundant k-mers. In this weighting the weight wₖₛw_{kz} grows logarithmically with the frequency fₖₛf_{kz}, reducing the effect on the score of highly abundant k-mers caused by sequencing artifacts.

Once their vectors have been constructed, the similarity between two samples (s₁s₁ and s₂s₂) is derived from the cosine of their vectors (vₛ₁vₛ₁ and vₛ₂vₛ₂) using following formula:

$$similarity ( {s}_{1} , {s}_{2} ) = cos ( {v}_{s 1} , {v}_{s 2} ) = \frac{{v}_{s 1} \cdot {v}_{s 2}}{‖ {v}_{s 1} ‖ \times ‖ {v}_{s 2} ‖} = \frac{{D}_{s 1 , s 2}}{{M}_{s 1} \times {M}_{s 2}} where , {D}_{s 1 s 2} = {v}_{s 1} \cdot {v}_{s 2} = \sum \text{?} w \text{?} \times w \text{?}$$

$$M \text{?} = ‖ v \text{?} ‖ = \sqrt{\sum \text{?} {( w \text{?} )}^{2}} \text{?}$$

$$\text{?} \text{indicates text missing or illegible when filed}$$

In other words, D_{s1,s2}D_{s1,s2} is the dot product of the vectors vₛ₁vₛ₁ and vₛ₂vₛ₂, M₂M₂ and is the magnitude (length) of the vector vₛvₛ. To score two samples S₁S₁ and S₂S₂, Libra computes the three values D_{s1,s2}D_{s1,s2}, Mₛ₁Mₛ₁, and Mₛ₂Mₛ₂. The values are calculated by scanning through the vectors vₛ₁vₛ₁ and vₛ₂vₛ₂ and computing the values. The computation time for scoring is proportional to the number of dimensions in the two vectors.

Although this technique allows Libra to compute the similarity between two vectors efficiently, it does not solve the problem of performing the all-vs-all comparisons efficiently. Mₛ is intrinsic to the vector vₛ and can be computed once and reused in all comparisons of vₛ with other vectors. However, D_{s1,s2}D_{s1,s2} is dependent on the pair vₛ₁vₛ₁ and vₛ₂vₛ₂ and is computed for each pair of vectors.

To address this issue, Libra performs a sweep line algorithm to compute the scores of all pairs in linear time. A sweep line algorithm or plane sweep algorithm is an algorithmic paradigm that uses a conceptual sweep line or sweep surface to solve various problems in Euclidean space. The sweep line algorithm only requires a single scan of all vectors to compute the similarity scores of all pairs of samples. It does this as follows (see, e.g., FIG. 17). Logically, Libra sweeps a line through all the vectors simultaneously starting with the first component. Libra outputs a record of the non-zero values of the following format:

record=k_mer:{<sample_id,weight>, <sample_id,weight>, . . . } Libra then moves the sweep line to the next component and performs the same operation. From the output records, contributions to MₛMₛ for each sample in the record are computed and accumulated. Contributions to DD are also computed from the record by extracting sample pairs. For example, the record {<s₁,s>, <s₂,y>, <s₄,z><s₁, x>, <s₂,y>, <s₄, z>} has three sample pairs (s₁s₂), (s₁s₄) and (s₂s₄)(s₁s₂), (s₁s₄) and (s₂s₄). Libra then computes contribution to DD for each pair, e.g. is x×yx×y is added to D_{s1,s2}D_{s1,s2}, x×zx×z is added to D_{s1,s4}D_{s1,s4}, and y×zy×z is added to D_{s2,s4}D_{s2,s4}. In this way, Libra computes similarity scores of every sample pairs in an input dataset in linear time.

One potential downside of this approach is that it requires n×(n−1)/2n×(n−1)/2 space to store the DD values. However, the required space is acceptable for reasonable values of nn on modern computing systems. Each value DD requires 8 bytes of memory so a dataset of 10000 samples requires only about 400 MB of memory.

Indexing.

An implementation based on the above description would require one vector per sample and the vectors would have 4k dimensions, where k is the k-mer length. If k is 20, for example, each vector would have more than one million dimensions. This is impractical, so Libra stores the k-mer frequencies from multiple samples in a single inverted index and performs the scoring on the index directly. The inverted index is indexed by k-mer, and contains the identifiers of the samples that contain that k-mer and its frequency in each sample. To be more precise, the key of the index is a k-mer and the value is a list of pairs, each of which contains a sample identifier and the frequency of the k-mer in the sample.
index record=k_mer:{<sample_id,frequency> . . . }
   The records in the index are stored in an alphabetical order by k-mer, allowing the record for a particular k-mer to be found via binary search. The k-mer record contains the k-mer frequency in each sample, not the weight, to allow for different weighting functions to be applied during scoring. The sweep algorithm is particularly easy to implement on an inverted index; it consists of simply stepping through the (sorted) k-mers. For each k-mer the index contains the frequency of that k-mer in each of the samples. Furthermore, the sweep algorithm is easily parallelized. The k-mer space is partitioned and a separate sweep is performed on each partition computing the contributions of its k-mer frequencies to the DD and MM values. At the end of the computation the intermediate DD and MM values are combined together to produce the final DD and MM values and thereby the distance matrix. Each sweep uses binary search to find the first k-mer in the partition.

Libra typically creates one index per group of samples. Libra groups samples automatically based on the number and size. If samples are too small, Libra groups them together to have a desired size (by default 4 GB per group). If the number of groups made is more than a threshold (20 by default), the groups are merged to reduce the number.

To construct an inverted index, Libra first generates the k-mers from the metagenomic samples at every offset, then converts them into a canonical representation. The canonical representation of a k-mer is either the forward form or the reverse complement form depending on which is first alphabetically. This allows the forward strand and the reverse strand of a k-mer to match.

An index is split into smaller chunks that are total-ordered so that the chunks can be constructed in parallel. A linear order, total order, simple order, or (non-strict) ordering is a binary relation on some set X, which is antisymmetric, transitive, and total (this relation is denoted here by infix ≤). A set paired with a total order is typically called a totally ordered set, a linearly ordered set, a simply ordered set, or a chain.

The index records are partitioned by k-mer ranges and the records in each partition is stored in a separate chunk file. All k-mers in partition n appear before the k-mers partition n+1 in lexicographic order. This breaks computation and IO down into smaller tasks, so that workload for creating an index can be distributed across several machines.

The range of k-mers for each partition is stored in a chunk index file. During scoring, the chunk index file is used to find the chunk file that contains a particular k-mer. Each sweep function finds the first k-mer in its partition by searching for the first possible k-mer in its partition. The chunk index file is used to find the proper chunk file, then binary search is used to look for the desired k-mer. If the first possible k-mer is not present, the next highest k-mer is used.

K-Mer Space Partitioning.

Both the index construction and the distance matrix computation require partitioning the k-mer space so that different partitions can be processed independently. For the partitioning to be effective, the workload should be balanced across the partitions. Simply partitioning into fixed-size partitions based on the k-mer space will not ensure balanced workloads, as the k-mers do not appear with uniform frequency. Some partitions may have more k-mer records than others, and thereby incur higher processing costs. Instead, the partitions should be created based on the k-mer distribution, so that each partition has roughly the same number of records. See FIG. 4 and FIG. 6, which are schema of the partitioning algorithm. Libra partitions the k-mer space based on the k-mer distribution to balance workloads across the partitions. Each partition has roughly the same number of records by having the same total k-mer counts. Computing the exact k-mer distribution across all the samples is too expensive in both space and time, therefore Libra approximates the distribution instead. A histogram is constructed using the first 6 letters of the k-mers in each sample, which requires much less space and time to compute. In practice, partitioning based on this histogram sufficiently partitions the k-mer space so that the workloads are adequately balanced across the partitions.

Libra Implementation.

Libra was implemented on the Hadoop MapReduce platform. This allows Libra to run on any standard Hadoop implementation (with MapReduce 2.0), while taking advantage of the scalability and fault-tolerance features provided by Hadoop. Hadoop allows robust parallel computation over distributed computing resources via its simple programming interface called MapReduce, while hiding much of the complexity of distributed computing (e.g. node failures). Taking advantage of Hadoop MapReduce, Libra can scale to larger input datasets and more computing resources. Furthermore, many cloud providers such as Amazon and Google, offer Hadoop clusters on a pay-as-you-go basis, allowing scientists to scale their Libra computations to match their datasets and budgets.

Libra is implemented using three different MapReduce jobs—1) k-mer histogram construction, 2) inverted index construction, and 3) distance matrix computation (scoring).

FIG. 1 shows a workflow of Libra.

Libra constructs a k-mer histogram of the input samples for load-balancing. A separate Map task is spawned for each input sample file that calculates the k-mer histogram for the sample. Alternatively, the k-mer histogram for a single sample could be computed using multiple Map tasks count k-mer frequencies in parallel and a Reduce task that combines their results. This approach, however, is inefficient because of the overhead required by Hadoop to manage multiple Map and Reduce tasks—a separate YARN container and a Java Virtual Machine (JVM) is spawned for each Map and Reduce task. Assigning a whole file to a Map task, however, eliminates the need for a Reduce task to combine the results. A downside of this approach is the workload for the k-mer histogram construction is distributed unevenly over the Hadoop cluster nodes as different sample files require different amounts of processing. Nevertheless, the unevenness is has little impact because each k-mer histogram construction task completes in a few minutes.

Libra performs the inverted index construction in parallel. In the Map phase, the work is split based on data blocks. A separate Map task is spawned for every data block in the input sample files. Each Map task generates k-mers from the sequences stored in a data block concurrently then passes them to the Reduce tasks. In the Reduce phase, the I/O and computation is split by partitioning the k-mer space using the k-mer histograms computed in the first phase. A separate Reduce task is spawned for every partition and a custom Partitioner routes the produced k-mers to Reduce tasks by their k-mer ranges. Each Reduce task then counts k-mers that passed in and produces an index chunk. As a result, each index chunk is stored as a separate file in the Hadoop MapFile format. The MapFile is well-suited for Libra as it is designed to store key-value pairs in key order, and allows binary search of the keys.

In the distance matrix computation, the work is split by partitioning the k-mer space in the beginning of a MapReduce job. The K-mer histogram files for input samples are loaded and merged, and the k-mer space is partitioned according to the k-mer distributions. A separate Map task is spawned for each partition to perform the computation in parallel. As a result, each task produces a JSON formatted output file containing partial contributions to the score matrix. At the end of the job, Libra merges the partial contributions from the files and produces the complete distance matrix.

### Results

Libra uses Hadoop MapReduce to perform massive all-vs-all sequence comparisons between next-generation sequence (NGS) datasets. Libra is designed to estimate genetic distance accurately without sacrificing performance. Instead, scalable algorithms and efficient resource usage make it feasible to perform all-vs-all comparisons on large datasets.

Libra performs all-vs-all distance comparisons using a sweep line algorithm. Naively, all-vs-all comparisons would require a total of n×(n−1)/2n×(n−1)/2 comparisons between nn samples. Using a sweep line algorithm,

Libra can perform these comparisons in a single pass. Libra maximizes cluster efficiency using a load balancing algorithm inspired by Terabyte Sort (O'Malley, et al., “O. Terabyte sort on apache hadoop,” Yahoo, Citeseer; 1-3 (2008)) to distribute the workload evenly over the Hadoop cluster. Highly parallelizable indexing and scoring algorithms enable Libra to scale to any size NGS dataset (often millions of reads), and perform any number of comparisons across datasets, making global ecosystem-level analyses possible.

### Example 2: Measuring Genetic Distance Between Simulated Metagenomes

### Materials and Methods

Staggered Mock Community.

Given that the above mixtures represented just two bacteria and most metagenomes are more complex, DNA from a staggered mock community obtained from the Human Microbiome Consortium were also sequenced. The staggered mock community is comprised of genomic DNA from a variety of genera commonly found on or within the human body, consisting of 1,000 to 1,000,000,000 16S rRNA gene copies per organism per aliquot. The resulting DNA was subjected to whole genome sequencing as described below under WGS sequencing. The sequence data comprised of ˜80 million reads have been deposited to the NCBI Sequence Read Archive under accession: SRP115095 under project accession PRJNA397434.

Simulated Data Derived from the Staggered Mock Community.

The resulting sequence data from the staggered mock community (˜80 million reads) were used to develop simulated metagenomes to test the effects of varying read depth, and composition and abundance of organisms in mixed metagenomes. To examine read depth (in terms of raw read counts and file size), the known staggered mock community abundance profile was used to generate an artificial metagenome using GemSim (McElroy, et al., BMC Genomics, 15; 13:74. doi: 10.1186/1471-2164-13-74 (2012)) of 1 million reads (454 sequencing) and duplicated the dataset 2× and 10×. The effects of sequencing a metagenome were simulated more deeply using GemSim (McElroy, et al., BMC Genomics, 15; 13:74. doi: 10.1186/1471-2164-13-74 (2012)) to generate simulated metagenomes with 0.5, 1, 5, and 10 million reads based on the relative abundance of organisms in the staggered mock community. Next, four simulated metagenomes were developed to test the effect of changing the dominant organism abundance and genetic composition including: 10 million reads from the staggered mock community (mock 1), the mock community with alterations in a few abundant species (mock 2), the mock community with many alterations in abundant species (mock 3), and mock 3 with additional sequences from archaea to further alter the genetic composition (mock 4) as illustrated in Table 2.

TABLE 2
Relative abundance of organisms in the mock communities.

| | relative abundance in the community | | | |
|---|---|---|---|---|
| genome_id | mock_1 | mock_2 | mock_3 | mock_4 |
| NC_002516.2 | 0.026955221 | 0.026955221 | 0.026955221 | 0.026955221 |
| NC_002695.1 | 0.155880357 | 0.002208926 | 0.002208926 | 0.002208926 |
| NC_009614.1 | 0.000168752 | 0.000168752 | 0.000168752 | 0.000168752 |
| NC_004350.2 | 0.214363721 | 0.015368135 | 0.015368135 | 0.015368135 |
| NC_004461.1 | 0.216517815 | 0.216517815 | 0.003648431 | 0.003648431 |
| NC_003098.1 | 0.00026295 | 0.00778332 | 0.00778332 | 0.00778332 |
| NC_004668.1 | 0.000275168 | 0.000275168 | 0.000275168 | 0.000275168 |
| NC_009428.1 | 0.312822437 | 0.312822437 | 0.02236441 | 0.02236441 |
| NC_001263.1 | 0.000344598 | 0.000344598 | 0.000344598 | 0.000344598 |
| GCA_000154225.1 | 0.000453573 | 0.000453573 | 0.000453573 | 0.000453573 |
| NC_004116.1 | 0.015368135 | 0.214363721 | 0.214363721 | 0.1 |
| NC_008530.1 | 0.001787557 | 0.001787557 | 0.001787557 | 0.001787557 |
| NC_003210.1 | 0.001805037 | 0.001805037 | 0.001805037 | 0.001805037 |
| NZ_CP009257.1 | 0.002208926 | 0.155880357 | 0.155880357 | 0 |
| NC_003112.2 | 0.00271185 | 0.00271185 | 0.00271185 | 0.00271185 |
| NC_006085.1 | 0.003648431 | 0.003648431 | 0.216517815 | 0 |
| NC_000915.1 | 0.005465696 | 0.005465696 | 0.005465696 | 0.005465696 |
| NZ_CP010086.2 | 0.00778332 | 0.00026295 | 0.00026295 | 0.00026295 |
| NC_004722.1 | 0.008812045 | 0.008812045 | 0.008812045 | 0.008812045 |
| NC_007795.1 | 0.02236441 | 0.02236441 | 0.312822437 | 0 |
| NC_009515.1 | 0 | 0 | 0 | 0.33 |
| NC_007681.1 | 0 | 0 | 0 | 0.2 |
| NZ_CP007536.1 | 0 | 0 | 0 | 0.269584331 |

Results

To test the effects of: (1) the size of the datasets, (2) depth of sequencing, (3) changing the abundance of dominant microbes in the community, and (4) changing the genetic composition of the community by adding in an entirely new organism (in this case archaea was added), simulated metagenomes were constructed and Libra's distance calculations were compared against those from Mash and SIMKA. Simulated datasets were derived from genomic DNA from a staggered mock community of bacteria that were obtained from the human microbiome consortium and sequenced deeply using the Ion Torrent sequencing platform (80 million reads, see methods). Libra uses a vector space model to compute the distance between two NGS datasets based on the composition of “words” or k-mers and their abundance in each sample. In this model each sample is represented by a vector, each dimension of which corresponds to a unique k-mer where the length and the angle of the vector relates to the abundance of the k-mer. The genetic distance between metagenomes is computed using the cosine of the angles between the vectors. By using the cosine distances, the size of the metagenome does not alter the resulting distances. This means that samples with different sequencing depths can be compared without additional normalization. Also, Libra allows users to select the optimal methodology for weighting high abundance k-mers in their datasets including boolean, natural logarithmic, and logarithmic. These options for weighting k-mers are important for different biological scenarios as described below using simulated datasets.

To demonstrate the cosine distance metric in Libra, simulated datasets derived from a mock bacterial community in known staggered abundance were examined In the first experiment, the effect of the size of the dataset was examined by using GemSim (McElroy, et al., BMC Genomics, 15; 13:74. doi: 10.1186/1471-2164-13-74 (2012)) to obtain a simulated metagenome composed of 1 million reads (454 sequencing) from the mock community and duplicating that dataset 2× and 10×. Overall, it was observed that altering the size of the metagenome (by duplicating the data) had no effect on the distance between metagenomes for Mash and Libra. In each case the distance of the duplicated datasets to the 1× mock community was less than 0.0001 (Table 3). SIMKA, however, did show a difference in the computed distance between duplicated datasets and the 1× mock community (Table 3) indicating that the metagenomes were not normalized as previously described (Benoit, et al., PeerJ Comput Sci. PeerJ Inc.; 2:e94 (2016)).

TABLE 3
Distance to 1x mock dataset using LIBRA, MASH
and SIMKA (Jaccard or Bray-Curtis distance)

| | | | SIMKA- | SIMKA - |
|---|---|---|---|---|
| | LIBRA | MASH | Jaccard | Bray Curtis |
| 1x mock1 | 0.00000 | 0 | 0 | 0 |
| 2x mock1 | 0.00000 | 0 | 0.351669 | 0.213349 |
| 10x mock1 | 0.00004 | 0 | 0.46696 | 0.304597 |

Because metagenomes don't scale exactly with size and instead have an increasing representation of low-abundance organisms, a second simulated dataset was created from the mock community using GemSim (McElroy, et al., BMC Genomics, 15; 13:74. doi: 10.1186/1471-2164-13-74 (2012)) with 0.5, 1, 5, and 10 million reads (454 sequencing) to mimic the effect of sequencing more deeply. Given the abundance of organisms in the mock community, the 0.5 M read dataset is mainly comprised of dominant species. With increased sequencing depth (1, 5, and 10 M reads) additional species are added relative to their abundance in the mock community. Overall, sequencing depth has little effect on the distance between samples in Mash and Libra (natural logarithmic weighting), whereas SIMKA shows a drastic change when using Jaccard and Bray-Curtis distances (FIG. 7A). These results indicate that Libra (natural logarithmic weighting) and Mash are appropriate for comparing datasets at different sequencing depths, whereas using SIMKA (Jaccard or Bray-Curtis) could lead to undesired effects. However SIMKA calculates many other distances that may be more suitable for this particular application.

In addition to natural variation in population-level abundances, artifacts from sequencing can result in high-abundance k-mers. Thus, when comparing the abundance of organisms in a sample, it is important to also be cautious of the effect of k-mers from artifacts compared to abundant species. To diminish the effect of high-abundance k-mers that may skew comparative analyses in Libra, a logarithmic weighting can be used. To examine the effect of weighting, the natural and logarithmic weight were compared and contrasted in Libra, with other distances obtained from Mash and SIMKA (Jaccard and Bray-Curtis). The effect of adding an entirely new species was also examined by spiking a simulated dataset with sequences derived from archaea (that were not present in the HMP staggered mock community). The simulated datasets were comprised of the staggered mock community (mock 1), the mock community with alterations in a few abundant species (mock 2), the mock community with many alterations in abundant species (mock 3), and mock 3 with additional sequences from archaea to alter the genetic composition of the community (mock 4) (see Table 2). The resulting data showed that Libra (logarithmic weighting) shows a stepwise increase in distance among the mock communities (FIG. 7B). This indicates that the logarithmic weighting in Libra allows for a comparison of distantly related microbial communities. Mash also shows a stepwise distance between communities, but is compressed relative to Libra, making differences less distinct. SIMKA (Bray-Curtis and Jaccard) and Libra (natural weighting) reach the maximum difference between mock communities 3 and 4 (FIG. 7B). This indicates that these distances are more appropriate when comparing metagenomes with small fluctuations in the community (for example, in a time-series analysis), whereas Libra (logarithmic weighting) can be used to distinguish metagenomes that vary in both genetic composition and abundance over a wide-range of species diversity, by dampening the effect of high-abundance k-mers. Because of this important difference, Libra was used with the logarithmic weighting in all subsequent analyses.

### Example 3: Libra can Distinguish Controlled Mixtures of Bacteria by Genetic Composition and Abundance

### Materials and Methods

Binary Mixtures of Bacteria.

To determine the sensitivity of Libra binary mixtures were created from purified bacterial DNA purchased from American Type Culture Collection (ATCC) isolated from: 1) Escherichia coli (ATCC 25922D-5) and Staphylococcus saprophyticus (15305D-5); 2) Streptococcus pyogenes (ATCC 12344D-5) and Staphylococcus saprophyticus (15305D-5); 3) Escherichia coli (ATCC 25922D-5) and Shigella flexneri (ATCC 29903D-5); and 4) methicillin-sensitive Staphylococcus aureus (MSSA, ATCC BAA-1718D-5) and methicillin-resistant S. aureus (MRSA, ATCC BAA-1717D-5). Bacterial mixtures represent phylogenetically diverse bacteria from least to most similar. DNA was resuspended in sterile phosphate buffered saline, quantitated from absorption at 260 nanometers using a NanoDrop ND-1000 spectrophotometer, and used to create binary mixtures of the following ratios by mass: 0.1:99.9, 1:99, 10:90, 50:50, 90:10, 99:1, 99.9:0.1. The resulting mixtures were subjected to whole genome sequencing as described below under WGS sequencing. The sequence data have been deposited to the NCBI Sequence Read Archive under accession: SRP116691 under project accession PRJNA401033.

Whole Genome Sequencing of Bacterial Mixtures and the Staggered Mock Community.

Mixtures were diluted to a final concentration of 1 nanogram/microliter and used to generate whole genome sequencing libraries with the Ion Xpress Plug Fragment Library Kit and manual #MAN0009847, revC (Thermo Fisher Scientific, Waltham, Mass., USA). Briefly, 10 nanograms of bacterial DNA was sheared using the Ion Shear enzymatic reaction for 12 min and Ion Xpress barcode adapters ligated following end repair. Following barcode ligation, libraries were amplified using the manufacturer's supplied Library Amplification primers and recommended conditions. Amplified libraries were size selected to ˜200 base pairs using the Invitrogen E-gel Size Select Agarose cassettes as outlined in the Ion Xpress manual and quantitated with the Ion Universal Library quantitation kit. Equimolar amounts of the library were added to an Ion PI Template OT2 200 kit V3. The resulting templated beads were enriched with the Ion OneTouch ES system and quantitated with the Qubit Ion Sphere Quality Control kit (Life Technologies) on a Qubit 3.0 fluorimeter (Qubit, NY, NY, USA). Enriched templated beads were loaded onto an Ion PI V2 chip and sequenced according to the manufacturer's protocol using the Ion PI Sequencing 200 kit V3 on a Ion Torrent Proton sequencer.

### Results

Determining the species composition of a sample is becoming an increasingly complex problem, as both the scale of NGS datasets and the number of reference genomes increase. Issues can also arise in distinguishing closely related species that vary by few but functionally important traits. For example, methicillin-sensitive vs -resistant Staphylococcus aureus (MSSA vs MRSA) share 97% of genomic content and differ by a single antibiotic-resistance cassette. Samples can also be polymicrobial with varied levels of microbial abundance, where rare species are difficult to detect given partial genomic content. As whole-genome shotgun sequencing becomes standard, building pipelines that are robust to both fine-scale differences in genomic content and abundance is vital.

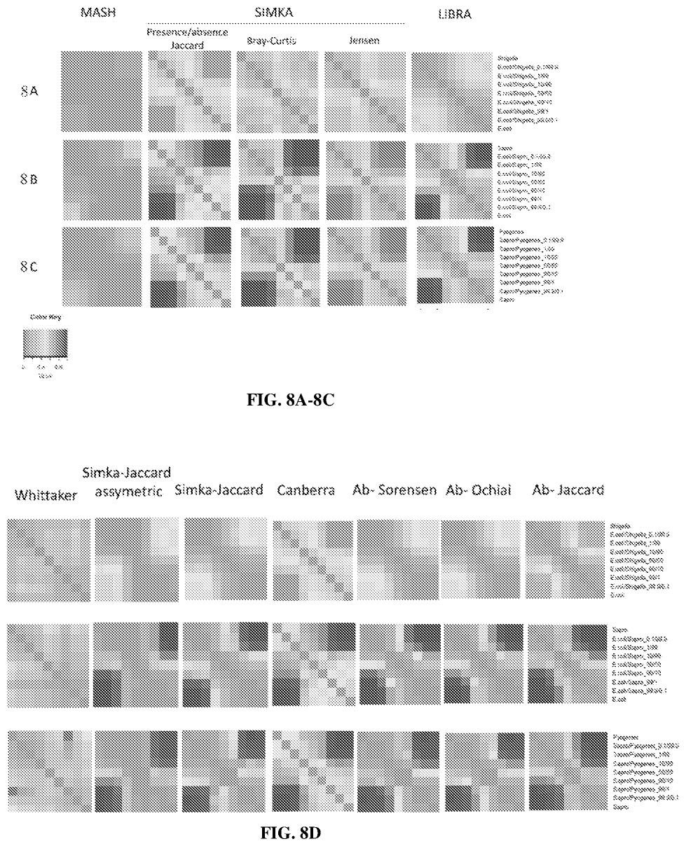

Four binary mixtures of bacterial species were created that vary by phylogenetic distance (from distant to closely related species) and abundance (across a six log range of dilution). Bacterial mixtures contained: 1) Gram-positive vs Gram-negative species (E. coli versus S. saprophyticus) that only share the same domain of life (bacteria); 2) Gram-positive species (S. saprophyticus vs S. pyogenes) that share the same class (Bacilli); 3) closely related bacteria (E. coli vs S. flexneri) from the same family (Enterobacteriaceae) that have divergent clinical impact; and 4) methicillin-sensitive vs -resistant Staphylococcus aureus (MSSA versus MRSA) that vary by a single antibiotic-resistance cassette. These data were used to compare and contrast capabilities in Mash, SIMKA, and Libra to distinguish mixtures by genomic composition and microbial abundance (FIG. 8A-8C). Importantly, each tool varies algorithmically based on how shared k-mers are computed (Mash computes on a subset of k-mers whereas SIMKA and Libra compute on all) and genetic distance metrics (Mash uses Jaccard presence/absence, SIMKA offers a variety of metrics, and Libra uses a vector-space model and cosine similarity). To compare each algorithmic choice independently, Mash and SIMKA were first compared using the Jaccard presence-absence distance metric to examine the effect of k-mer sub-sampling (FIG. 8A-8C). Next, quantitative distance metrics were compared using SIMKA and Libra to test capabilities in capturing microbial abundance (FIG. 8A-8D)

When comparing Mash and SIMKA using the Jaccard presence-absence distance metric, two clear patterns emerged (FIG. 8A-8C). First, subsampling using Mash (even when increasing to a sketch size of 10k k-mers from the 1k default setting) greatly reduces the detectable genetic distance among all samples from phylogenetically diverse to closely related bacteria. Secondly, although SIMKA shows increased resolution by using all k-mers, the Jaccard presence-absence distance metric cannot recapitulate the logarithmic scale of bacterial abundance. Instead, mixtures are roughly defined as a single organism for the nearly pure mixtures at 99.9% and 99% (FIG. 8A-8C). Overall, sub-sampling greatly reduces the computed genetic distance, and the Jaccard presence-absence metric is unable to distinguish differences in abundance in bacterial mixtures.

Next, quantitative distance metrics in SIMKA were examined FIG. 8A-8C: Bray-Curtis and Jensen, 8D and Libra (vector-space model with cosine-similarity) using all k-mers for each dataset. In the most phylogenetically distant pairing (E. coli vs S. saprophyticus), both Libra and SIMKA (Jensen) were able to separate mixtures by organism according to their log-based composition (FIG. 8A). SIMKA (Bray-Curtis) separated samples by the most abundant organism but was unable to distinguish log-based differences in microbial abundance. Further, distances computed using Bray-Curtis varied in the second half of the matrix and may indicate classification errors. As the bacterial mixtures became increasingly phylogenetically similar to one another (FIGS. 8B and 8C), both Libra and SIMKA (Jensen) were able to accurately compute differences by both genetic distance and microbial abundance. Interestingly, Libra shows a cleaner separation at closer ratios (90:10) than SIMKA (Jensen).

In the final mixture containing methicillin-sensitive vs -resistant Staphylococcus aureus (MSSA vs MRSA), only Libra and SIMKA (Jensen) were able to distinguish the strains (Table 4). MRSA contains an antibiotic resistance gene called mecA that is embedded in a chromosomal cassette called the SCC mec element. The overall genetic distance between strains is negligible (97.4% of the MRSA genome is shared with MSSA), demonstrating that only Libra and SIMKA (Jensen) can detect fine-scale strain-level differences.

TABLE 4
Comparison of various metrics (similarity scores) for
the analysis of a binary mixture of MSSA and MRSA.

| | SIMKA | | SIMKA | | SIMKA | | | |
|---|---|---|---|---|---|---|---|---|
| | Jaccard | | Bray-Curtis | | Jensen | | Libra | |
| MSSA/MRSA | MSSA | MRSA | MSSA | MRSA | MSSA | MRSA | MSSA | MRSA |
| 0.10/99.9 | 0.599 | 0.537 | 0.749 | 0.699 | 0.739 | 0.794 | 0.852 | 0.875 |
| 1.0/99 | 0.609 | 0.555 | 0.757 | 0.714 | 0.736 | 0.791 | 0.852 | 0.875 |
| 10/90 | 0.649 | 0.626 | 0.787 | 0.770 | 0.744 | 0.790 | 0.867 | 0.884 |
| 50/50 | 0.656 | 0.590 | 0.792 | 0.742 | 0.764 | 0.776 | 0.865 | 0.876 |
| 90/10 | 0.681 | 0.626 | 0.810 | 0.770 | 0.787 | 0.747 | 0.884 | 0.882 |
| 99/1.0 | 0.676 | 0.620 | 0.807 | 0.766 | 0.790 | 0.729 | 0.889 | 0.870 |
| 99.9/0.10 | 0.701 | 0.618 | 0.824 | 0.764 | 0.799 | 0.734 | 0.896 | 0.874 |

Highlighted in bold, the highest similarity score obtained when compared to pure MSSA or MRSA sample.

Based on the analyses of controlled binary mixtures of bacteria, Libra and SIMKA (Jensen) are effective at separating samples by genetic distance and microbial abundance. However, the two algorithms are implemented in different ways that affect complexity and speed. Both SIMKA and Libra construct k-mer indices from reads in input samples to simplify scoring. SIMKA constructs a massive k-mer index for all samples that requires a merge in indexing that can compromise the speed and scalability of the algorithm. Whereas, Libra performs scoring directly over multiple k-mer indices without an additional merge, offering better reusability of pre-constructed indices and the ability to scale to any size dataset. Libra is designed to perform on a Hadoop cluster while SIMKA runs on a HPC cluster. Relying on different base systems, the two algorithms result in different runtimes (speed), scalability, fault-tolerance, and accessibility. According to the published metrics, it is seems that SIMKA performs faster than Libra. However, these calculations do not take into account additional overhead in resource and task management in Hadoop (that Libra is based on) that guarantee scalability and fault-tolerance for massive datasets. For this reason, Hadoop clusters are now available in a variety of settings including academic science clouds (e.g., Wrangler at TACC (Devisetty, et al., CyVerse Discovery Environment, 5:1442, F1000Res. (2016)) and commercial clouds (e.g., Amazon EMR (Amazon EMR—Amazon Web Services [Internet]. [cited 20 Dec. 2017]). Because the runtime for SIMKA (Jensen) increases quadratically as the number of samples increases (Benoit, et al., PeerJ Comput Sci. PeerJ Inc.; 2:e94 (2016)), analyses was limited to just Libra and Mash for large-scale datasets below including the human microbiome project (HMP) and Tara Oceans Expedition.

### Example 4: Profiling Differences in Bacterial Diversity and Abundance in the Human Microbiome

### Materials and Methods

Human Microbiome 16S rRNA Gene Amplicons and WGS Reads.

Human microbiome datasets were downloaded from the NIH Human microbiome project (Human Microbiome Project Consortium, Nature, 486(7402):207-14. doi: 10.1038/nature11234 (2012)) including 48 samples from 5 body sites including: urogenital (posterior fomix), gastrointestinal (stool), oral (buccal mucosa, supragingival plaque, tongue dorsum), airways (anterior nares), and skin (retroauricular crease left and right) (See Table 5). Matched datasets consisting of 16S rRNA reads, WGS reads, and WGS assembled contigs were downloaded from the 16S trimmed dataset and the HMIWGS/HMASM dataset respectively.

TABLE 5
HMP samples metadata

| | Sex | HMPBodySubsite | HMPBodySite |
|---|---|---|---|
| SRS015450 | male | Anterior_nares | Airways |
| SRS016434 | male | Anterior_nares | Airways |
| SRS016553 | female | Anterior_nares | Airways |
| SRS017044 | male | Anterior_nares | Airways |
| SRS018463 | male | Anterior_nares | Airways |
| SRS018585 | male | Anterior_nares | Airways |
| SRS013506 | male | Buccal_mucosa | Oral |
| SRS013711 | male | Buccal_mucosa | Oral |
| SRS013825 | male | Buccal_mucosa | Oral |
| SRS015921 | male | Buccal_mucosa | Oral |
| SRS016349 | male | Buccal_mucosa | Oral |
| SRS016533 | female | Buccal_mucosa | Oral |
| SRS013258 | male | Left_Retroauricular_crease | Skin |
| SRS016944 | male | Left_Retroauricular_crease | Skin |
| SRS017849 | male | Left_Retroauricular_crease | Skin |
| SRS020261 | male | Left_Retroauricular_crease | Skin |
| SRS024482 | male | Left_Retroauricular_crease | Skin |
| SRS024596 | male | Left_Retroauricular_crease | Skin |
| SRS011584 | female | Posterior_fornix | Urogenital_tract |
| SRS014343 | female | Posterior_fornix | Urogenital_tract |
| SRS014629 | female | Posterior_fornix | Urogenital_tract |
| SRS015425 | female | Posterior_fornix | Urogenital_tract |
| SRS016111 | female | Posterior_fornix | Urogenital_tract |
| SRS016559 | female | Posterior_fornix | Urogenital_tract |
| SRS013261 | male | Right_Retroauricular_crease | Skin |
| SRS017851 | male | Right_Retroauricular_crease | Skin |
| SRS020263 | male | Right_Retroauricular_crease | Skin |
| SRS024598 | male | Right_Retroauricular_crease | Skin |
| SRS024655 | male | Right_Retroauricular_crease | Skin |
| SRS019081 | male | Right_Retroauricular_crease | Skin |
| SRS011271 | male | Stool | Gastrointestinal_tract |
| SRS011405 | female | Stool | Gastrointestinal_tract |
| SRS011452 | male | Stool | Gastrointestinal_tract |
| SRS016954 | male | Stool | Gastrointestinal_tract |
| SRS019910 | male | Stool | Gastrointestinal_tract |
| SRS023176 | male | Stool | Gastrointestinal_tract |
| SRS013252 | male | Supragingival_plaque | Oral |
| SRS013723 | male | Supragingival_plaque | Oral |
| SRS015470 | male | Supragingival_plaque | Oral |
| SRS015574 | male | Supragingival_plaque | Oral |
| SRS016331 | male | Supragingival_plaque | Oral |
| SRS016541 | female | Supragingival_plaque | Oral |
| SRS013234 | male | Tongue_dorsum | Oral |
| SRS013502 | male | Tongue_dorsum | Oral |
| SRS013705 | male | Tongue_dorsum | Oral |
| SRS014271 | male | Tongue_dorsum | Oral |
| SRS015395 | female | Tongue_dorsum | Oral |
| SRS015762 | male | Tongue_dorsum | Oral |

### Results

Microbial diversity is traditionally assessed using two methods: the 16S rRNA gene to classify bacterial and archaeal groups at the genus level, or whole genome shotgun sequencing (WGS) for finer taxonomic classification at the species or subspecies level. Further, WGS datasets provide additional information on functional differences between metagenomes. The effect of different algorithmic approaches (Mash vs Libra), data type (16S rRNA vs WGS), and sequence type (WGS reads vs assembled contigs) were compared and contrasted in analyzing data from 48 samples across 8 body sites from the Human Microbiome Project. Specifically, matched datasets (16S rRNA reads, WGS reads, and WGS assembled contigs) classified as urogenital (posterior fomix), gastrointestinal (stool), oral (buccal mucosa, supragingival plaque, tongue dorsum), airways (anterior nares), and skin (retroauricular crease left and right) (Table 5) were examined

Because the HMP datasets represent microbial communities, abundant bacteria will have more total read counts than rare bacteria in the samples. Thus, each sample can vary by both taxonomic composition (the genetic content of taxa in a sample) and abundance (the relative proportion of those taxa in the samples). Importantly, the 16S rRNA amplicon dataset is useful in showing how well each algorithm performs in detecting and quantifying small-scale variation for a single gene at the genus-level, whereas the WGS dataset demonstrates the effect of including the complete genetic content and abundance of organisms at the species-level in a community (Watts, et al, Wiley Online Library, 123:1584-1596 (2017)). Also, differences in each algorithm were examined when read abundance is excluded using assembled contigs that only represent the genetic composition of the community.

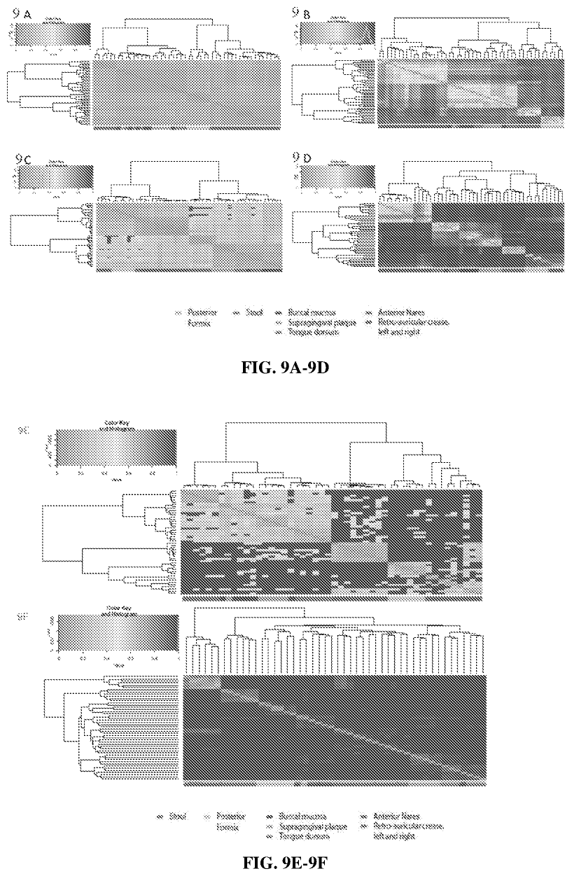

Using the 16S rRNA reads, both Mash and Libra clustered samples by broad categories but not individual body-sites (FIGS. 9A and 9B). Similar to what is described in previous work (Benoit, et al., PeerJ Comput Sci. PeerJ Inc.; 2:e94 (2016)), samples from the airways and skin co-cluster, whereas other categories including urogenital, gastrointestinal, and oral are distinct (Benoit, et al., PeerJ Comput Sci. PeerJ Inc.; 2:e94 (2016)). These results indicate that limited variation in the 16S rRNA gene may only allow for clustering for broad categories. Further, the Mash algorithm shows lower overall resolution (FIG. 9A) as compared to Libra (FIG. 9B). Indeed, amplicon sequencing analysis is not an original intended use of Mash, given that it reduces the dimensionality of the data by looking at presence/absence of unique k-mers, whereas Libra examines the complete dataset accounting for both composition in organisms and their abundance. When using WGS reads, both Mash and Libra show enhanced clustering by body-site (FIGS. 9C and 9D), however Mash shows decreased resolution (FIG. 9C) as compared to Libra (FIG. 9D). Again, these differences reflect the effect of using all of the read data (Libra) rather than a subset (Mash). Importantly, the Libra algorithm also depends on read abundance that provides increased resolution for interpersonal variation as seen in skin samples (FIG. 9D). When abundance is taken out of the equation by using assembled contigs (FIGS. 9E and 9F) Mash performs well in clustering distinct body sites whereas Libra shows discrepancies and less overall resolution. Thus, for Libra to perform accurately and in order to obtain a high-resolution clustering, Libra should be run on reads rather than contigs (FIG. 9D).

### Example 5: Ecosystem-Scale Analyses: Clustering Marine Viromes to Examine Global Diversity

### Materials and Methods

Tara Ocean Viromes.

Tara oceans viromes were downloaded from European Nucleotide Archive (ENA) at EMBL and consisted of 43 viromes from 43 samples at 26 locations across the world's oceans collected during the Tara Oceans (2009-2012) scientific expedition (Brum, et al., Science, 348, doi:10.1126/science.1261498 (2015)). Metadata for the samples was downloaded from PANGAEA (Diepenbroek, et al., Comput Geosci. Elsevier, 28:1201-1210 (2002)). These samples were derived from multiple depths including: 16 surface samples (5-6 meters), 18 deep chlorophyll maximum samples (DCM; 17-148 meters), and one mesopelagic sample (791 meters). Quality control procedures were applied according to methods described by Brum and colleagues (Brum, et al., Science, 348, doi:10.1126/science.1261498 (2015)) (Table 6).

TABLE 6
Tara Ocean viromes metadata

| | | Depth | | | | Prov- | | | |
|---|---|---|---|---|---|---|---|---|---|
| Name | Station | category | Mean_Date | Mean_Lat | Mean_Long | ince | Full Name | Biome | Mean_Depth |
| C1_station18, | 18_DCM | DCM | Nov. 2, 2009 12:43 | 35.76055 | 14.25215 | MEDI | Mediterranean | Westerlies | 61 |
| DCM | | | | | | | Sea, | | |
| C1_station18, | 18_SUR | surface | Nov. 2, 2009 8:18 | 35.75773 | 14.260883 | MEDI | Mediterranean | Westerlies | 5 |
| SUR | | | | | | | Sea, | | |
| C1_station23, | 23_DCM | DCM | Nov. 18, 2009 15:32 | 42.16131 | 17.731333 | MEDI | Mediterranean | Westerlies | 55 |
| DCM | | | | | | | Sea, | | |
| C1_station25, | 25_DCM | DCM | Nov. 23, 2009 12:40 | 39.40855 | 19.380733 | MEDI | Mediterranean | Westerlies | 51 |
| DCM | | | | | | | Sea, | | |
| C1_station25, | 25_SUR | surface | Nov. 23, 2009 9:14 | 39.38772 | 19.390967 | MEDI | Mediterranean | Westerlies | 5 |
| SUR | | | | | | | Sea, | | |
| C1_station30, | 30_DCM | DCM | Dec. 15, 2009 16:17 | 33.92529 | 32.774146 | MEDI | Mediterranean | Westerlies | 69 |
| DCM | | | | | | | Sea, | | |
| W0_station31, | 31_SUR | surface | Jan. 9, 2010 8:43 | 27.15 | 34.818333 | REDS | Red Sea, | Coastal | 5 |
| SUR | | | | | | | Persian | | |
| W0_station31, | 32_DCM | DCM | Jan. 11, 2010 14:27 | 23.38333 | 37.2525 | REDS | Red Sea, | Coastal | 81 |
| DCM | | | | | | | Persian | | |
| W0_station32, | 32_SUR | surface | Jan. 11, 2010 8:33 | 23.37333 | 37.215833 | REDS | Red Sea, | Coastal | 5 |
| SUR | | | | | | | Persian | | |
| W0_station34, | 34_DCM | DCM | Jan. 20, 2010 7:44 | 18.39833 | 39.861667 | REDS | Red Sea, | Coastal | 60 |
| DCM | | | | | | | Persian | | |
| W0_station34, | 34_SUR | surface | Jan. 20, 2010 6:17 | 18.3975 | 39.869167 | REDS | Red Sea, | Coastal | 5 |
| SUR | | | | | | | Persian | | |
| W1_station36, | 36_DCM | DCM | Mar. 12, 2010 10:34 | 20.81755 | 63.512117 | ARAB | NW | Coastal | 17 |
| DCM | | | | | | | Arabian | | |
| W1_station36, | 36_SUR | surface | Mar. 12, 2010 6:19 | 20.81833 | 63.50465 | ARAB | NW | Coastal | 5 |
| SUR | | | | | | | Arabian | | |
| W0_station41, | 41_DCM | DCM | Mar. 30, 2010 11:41 | 14.5636 | 70.039808 | MONS | Indian | Trades | 59 |
| DCM | | | | | | | Monsoon | | |
| W0_station41, | 41_SUR | surface | Mar. 30, 2010 2:56 | 14.5954 | 69.981 | MONS | Indian | Trades | 5 |
| SUR | | | | | | | Monsoon | | |
| W0_station42, | 42_DCM | DCM | Apr. 4, 2010 11:34 | 5.992733 | 73.9045 | MONS | Indian | Trades | 79 |
| DCM | | | | | | | Monsoon | | |
| W0_station42, | 42_SUR | surface | Apr. 4, 2010 3:13 | 6.031333 | 73.89415 | MONS | Indian | Trades | 5 |
| SUR | | | | | | | Monsoon | | |
| W0_station46, | 46_SUR | surface | Apr. 15, 2010 2:38 | −0.66245 | 73.160967 | MONS | Indian | Trades | 5 |
| SUR | | | | | | | Monsoon | | |
| W1_station52, | 52_DCM | DCM | May 17, 2010 12:37 | −16.95683 | 54.010317 | ISSG | Indian S. | Westerlies | 77 |
| DCM | | | | | | | Subtropical | | |
| W1_station64, | 64_DCM | DCM | Jul. 8, 2010 5:19 | −29.54357 | 37.9356 | ISSG | Indian S. | Westerlies | 63 |
| DCM | | | | | | | Subtropical | | |
| W1_station64, | 64_SUR | surface | Jul. 7, 2010 4:56 | −29.50222 | 37.9904 | ISSG | Indian S. | Westerlies | 5 |
| SUR | | | | | | | Subtropical | | |
| C1_station66, | 66_DCM | DCM | Jul. 15, 2010 16:32 | −34.89331 | 18.072967 | EAFR | E. Africa | Coastal | 28 |
| DCM | | | | | | | Coastal | | |
| C1_station66, | 66_SUR | surface | Jul. 15, 2010 12:26 | −34.93627 | 17.936583 | EAFR | E. Africa | Coastal | 5 |
| SUR | | | | | | | Coastal | | |
| C1_station68, | 68_DCM | DCM | Sep. 14, 2010 9:14 | −31.0302 | 4.687867 | SATL | South | Westerlies | 40 |
| DCM | | | | | | | Atlantic | | |
| C1_station68, | 68_SUR | surface | Sep. 14, 2010 9:14 | −31.0302 | 4.687867 | SATL | South | Westerlies | 5 |
| SUR | | | | | | | Atlantic | | |
| C0_station70, | 70_MES | meso- | Sep. 22, 2010 10:38 | −20.36694 | −3.217333 | SATL | South | Westerlies | 791 |
| MES | | pelagic | | | | | Atlantic | | |
| C0_station70, | 70_SUR | surface | Sep. 21, 2010 7:05 | −20.43683 | −3.18585 | SATL | South | Westerlies | 5 |
| SUR | | | | | | | Atlantic | | |
| W1_station72, | 72_DCM | DCM | Oct. 5, 2010 12:42 | −8.702583 | −17.93975 | SATL | South | Westerlies | 95 |
| DCM | | | | | | | Atlantic | | |
| W1_station72, | 72_SUR | surface | Oct. 5, 2010 8:10 | −8.776367 | −17.912733 | SATL | South | Westerlies | 5 |
| SUR | | | | | | | Atlantic | | |
| W1_station76, | 76_DCM | DCM | Oct. 16, 2010 17:43 | −21.04563 | −35.368772 | SATL | South | Westerlies | 147 |
| DCM | | | | | | | Atlantic | | |
| W1_station76, | 76_SUR | surface | Oct. 16, 2010 10:07 | −20.94063 | −35.195967 | SATL | South | Westerlies | 5 |
| SUR | | | | | | | Atlantic | | |
| C0_station82, | 82_DCM | DCM | Dec. 6, 2010 22:04 | −47.16529 | −57.895717 | FKLD | SW | Coastal | 41 |
| DCM | | | | | | | Atlantic | | |
| C0_station85, | 85_DCM | DCM | Jan. 6, 2011 20:36 | −62.24368 | −49.182663 | APLR | Austral | Polar | 87 |
| DCM | | | | | | | Polar | | |
| W0_station109, | 109_DCM | DCM | May 13, 2011 0:00 | 2.076667 | −84.520283 | PNEC | N. Pacific | Trades | 29 |
| DC | | | | | | | Equatorial | | |
| W0_station109, | 109_SUR | surface | May 12, 2011 16:15 | 2.017525 | −84.573308 | PNEC | N. Pacific | Trades | 5 |
| SUR | | | | | | | Equatorial | | |
| C0_station67, | 67_SUR | surface | Sep. 7, 2010 6:19 | −32.2401 | 17.7103 | BENG | South | Coastal | 5 |
| SUR | | | | | | | Atlantic | | |
| C1_station22, | 22_SUR | surface | Nov. 16, 2010 8:16 | 39.8386 | 17.4155 | MEDI | Mediterranean | Westerlies | 5 |
| SUR | | | | | | | Sea, | | |
| W0_station38, | 38_SUR | surface | Mar. 16, 2010 6:13 | 19.0393 | 64.4913 | MONS | Indian | Trades | 5 |
| SUR | | | | | | | Monsoon | | |
| W0_station38, | 38_DCM | DCM | Mar. 15, 2010 11:18 | 19.0284 | 64.5126 | MONS | Indian | Trades | 25 |
| DCM | | | | | | | Monsoon | | |
| W0_station39, | 39_SUR | surface | Mar. 18, 2010 4:27 | 18.5918 | 66.622 | MONS | Indian | Trades | 5 |
| SUR | | | | | | | Monsoon | | |
| W0_station39, | 39_DCM | DCM | Mar. 18, 2010 11:33 | 18.5839 | 66.4727 | MONS | Indian | Trades | 25 |
| DCM | | | | | | | Monsoon | | |
| W1_station65, | 65_SUR | surface | Jul. 12, 2010 5:59 | −35.1728 | 26.2868 | EAFR | E. Africa | Coastal | 5 |
| SUR | | | | | | | Coastal | | |
| W1_station65, | 65_DCM | DCM | Jul. 12, 2010 11:03 | −35.2421 | 26.3048 | EAFR | E. Africa | Coastal | 30 |
| DCM | | | | | | | Coastal | | |

### Results

To demonstrate the scale and performance of the Libra algorithm, 43 Tara Ocean Viromes (TOV) from the 2009-2011 Expedition (Brum, et al., Science, 348, doi:10.1126/science.1261498 (2015)) representing 26 sites, 43 samples, and 4.2 billion reads from the global ocean (Table 7) were analyzed. Phages (viruses that infect bacteria) are abundant in the ocean (Bergh, et al., Nature, 340:467-468 (1989)) and can significantly impact environmental processes through host mortality, horizontal gene transfer, and host-gene expression. Yet, how phages change over space and time in the global ocean and with environmental fluxes is just beginning to be explored. The primary challenge is the majority of reads in viromes (often >90%) do not match known proteins or viral genomes (Hurwitz, et al., PLoS One, 8:e57355 (2013)) and no conserved genes like the bacterial 16S rRNA gene exist to differentiate populations. To examine known and unknown viruses simultaneously, viromes are best compared using sequence signatures to identify common viral populations.

TABLE 7
Statistics of the Tara Ocean Viromes (TOV) dataset.

| | | | |
|---|---|---|---|
| Total # of | 43 | Total # of | 62 (124 |
| viromes | | virome sequencing | paired-end |
| | | runs | files) |
| Total size of | 551.6 GB | Mean size of each | 4.4 GB |
| all virome | | virome paired-end | |
| files | | sequencing run | |
| Total # of | 4,194,402,268 | Mean # of reads | 33,555,218.14 |
| reads | | in each virome | |
| | | paired-end | |
| | | sequencing run | |
| Total # of | 403,891,365,808 | Mean # of base | 3,231,130,926 |
| base pairs | | pairs in each | |
| (bp) | | virome paired-end | |
| | | sequencing run | |
| | | Mean read length | 96.29 |

Two approaches exist to cluster viromes based on sequence composition. The first approach uses protein clustering to examine functional diversity in viromes between sites (Brum, et al., Science, 348, doi:10.1126/science.1261498 (2015); Hurwitz, et al., PLoS One, 8:e57355 (2013); Hurwitz, et al., ISME J., 9(2):472-84 (2015), Epub 2014 Aug. 5). Protein clustering, however, depends on accurate assembly and gene finding that can be problematic in fragmented and genetically diverse viromes (Minot, et al., PLoS One, 7: e42342 (2012)). Further, assemblies from viromes often only include a fraction of the total reads (e.g., only ¼ in TOV (Brum, et al., Science, 348, doi:10.1126/science.1261498 (2015)). To examine global viral diversity in the ocean using all of the reads TOV was examined using Libra. The complete pairwise analysis of ˜4.2 billion reads in the TOV dataset (Brum, et al., Science, 348, doi:10.1126/science.1261498 (2015)) finished in 18 hours using a 10-node Hadoop cluster (see Methods and Table 8). Importantly, Libra exhibits remarkable performance in computing similarity scores, wherein k-mer matches for all TOV completed within 1.5 hours (Table 8). This step usually represents the largest computational bottleneck for bioinformatics tools that compute pairwise distances between sequence pairs for applications such as hierarchical sequence clustering (Edgar, et al., BMC Bioinformatics, 26:2460-2461 (2010); Sun, et al., Oxford Univ Press, 13:107-121 (2012); Niu, et al., BMC Bioinformatics, 11:187 (2010); Cai, et al., Oxford Univ Press, 39:e95 (2011)).

TABLE 8
Execution times for the Libra based on
the Tara Ocean Virome (TOV) dataset.

| Stage | Execution Time |
|---|---|
| Indexing | 16:32:55 |
| Scoring | 1:24:27 |
| Total | 17:57:22 |

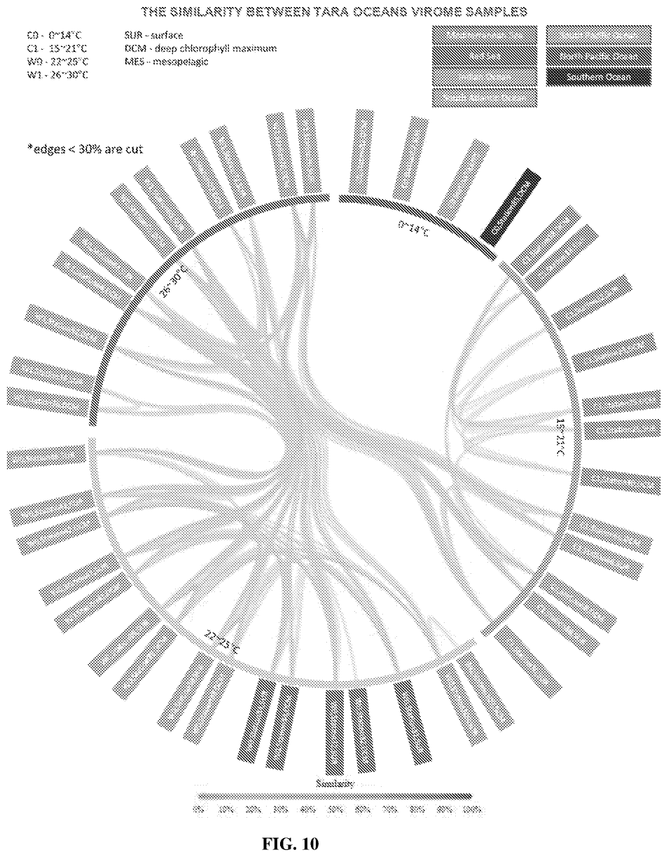

Overall, viral populations in the ocean are largely structured by temperature in four gradients (FIG. 10) similar to their bacterial hosts (Sunagawa, et al., Science, 348(6237):1261359, doi:10.1126/science.1261359 (2015)). Interestingly, samples from different Longhurst Provinces but the same temperature gradient cluster together. Also, water samples from the surface (SUR) and deep chlorophyll maximum (DCM) at the same station, cluster more closely together than samples from the same depth at nearby sites (FIG. 10). These data indicate that viral populations are structured globally by temperature, and at finer resolution by station (for surface and DCM samples) indicating that micronutrients and local conditions play an important role in viral populations. Also noteworthy, samples that were derived from extremely cold environments (noted as C0 in FIG. 10) lacked similarity to all other samples (at a 30% similarity score), indicating distinctly different viral populations. These samples include a mesotrophic sample that have previously been shown to have distinctly different viral populations than surface ocean samples (Hurwitz, et al., ISME J., 9:472-484 (2015)). These data contrast prior work (Brum, et al., Science, 348, doi:10.1126/science.1261498 (2015); Breitbart, et al., Trends Microbiol, 13:278-284 (2005)) that argues for the seed bank hypothesis where viral populations exist globally but vary in abundance based on host abundance and distribution. Instead, it is possible that viral populations can differ by location and are defined by temperature and small-scale differences in local environments.

### Example 6: Tuning a Benchmark Environment

The experiments below were performed on a Hadoop cluster consisting of 10 physical nodes (9 MapReduce worker nodes). Each node contains 12 Intel Xeon X5670 CPUs each runs at 2.93 GHz and 128 GB of RAM, and is configured to run a maximum of 7 YARN containers simultaneously with 10 GB of RAM per container. The remaining system resources are reserved for the operating system and other Hadoop services.

To demonstrate the scale and performance of the Libra algorithm, 43 Tara Ocean Viromes (TOV) from the 2009-2011 Expedition (Brum, et al., Science, 348:6237 (2015)), representing 26 sites, 43 samples, 4.2 billion reads and 324 billion k-mers from the global ocean (Table 1) were analyzed.

To demonstrate the advantages of Libra's load-balancing, a subset of the Tara Ocean Viromes (TOV) that consists of 10 random samples in a total of 119.2 GB was used for all the load-balancing benchmarks. Several changes were made to Libra's configuration to facilitate accurate measurements. First, during the inverted index construction Hadoop was configured to not run the Reduce tasks until the Map tasks completed (i.e. Reduce tasks could not overlap Map tasks). By default, Hadoop will start some Reduce tasks before the Map tasks complete, causing the Reduce tasks to wait for input and disturbing the measurements. Second, Libra was configured to only use a single group for the input samples. Without this, Libra would produce groups of different sizes, making it difficult to measure workload imbalances.

In all experiments Libra was configured to have 256 partitions. Therefore, 256 Reduce tasks were created during the inverted index construction, 256 index chunks were constructed and 256 map tasks were created during the distance matrix computation. The same benchmark was performed with the same samples three times and the results averaged.

Workload Distribution in the Inverted Index Construction Input-Splitting

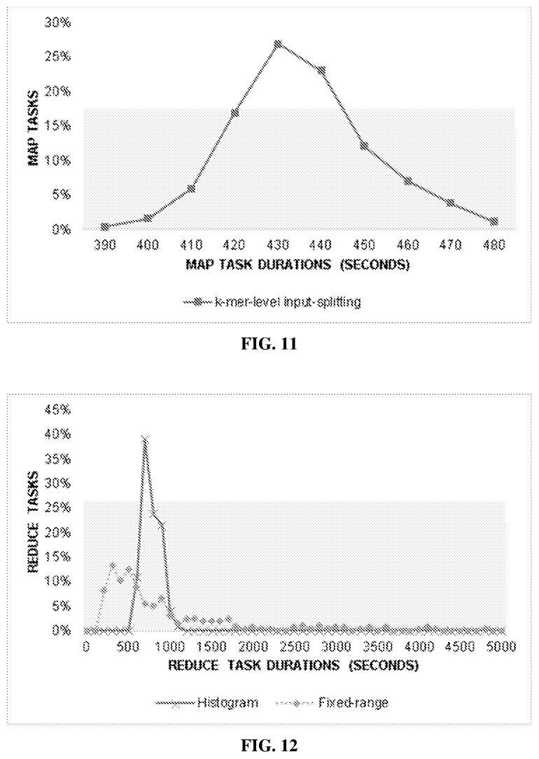

The k-mer-level input splitting algorithm balanced the durations of the Map tasks during the inverted index construction (k-mer counting) as shown in FIG. 11. Approximately 80% of Map tasks completed in 420˜450 seconds. There were very few tasks with relatively short durations (<170 seconds). This was caused by partial input splits at the end of the sample files, but they did not affect the overall results.

Partitioning

The partitioning algorithm was compared with the fixed-range partitioning algorithm because their inverted indices produced have entries in the same order, in the lexicographic order of k-mers. This meets the goals for allowing reuse of indices at different machines and different analysis without merging indices.

FIG. 12 shows the durations of Reduce tasks in the inverted index construction using different partitioning algorithms Using the histogram-based partitioning 100% of Reduce tasks completed in less than 1100 seconds, and 84% of tasks completed in 700 to 900 seconds. In comparison, fixed-range partitioning had a much wider distribution of durations, with 70% of tasks finishing in less than 900 seconds, but with a long tail out to 4800 seconds. This wider distribution of durations leads to larger load imbalances between the Reduce tasks, leading to larger durations of the overall computation.

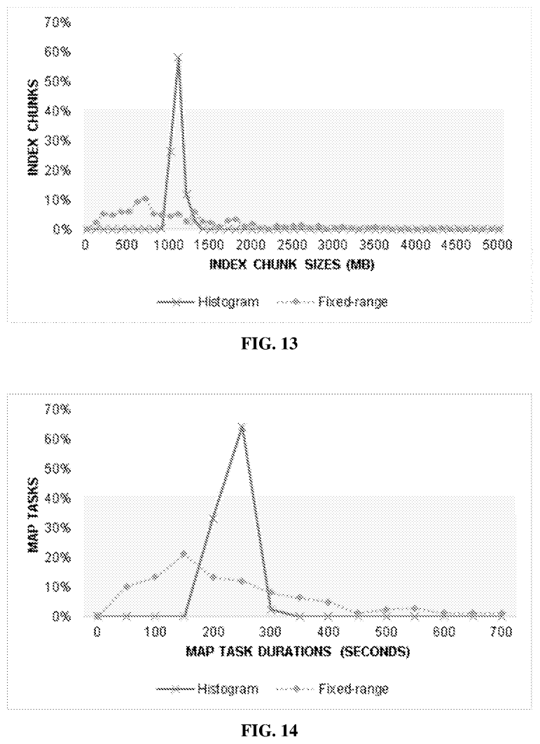

FIG. 13 shows the sizes of the index chunks produced by the Reduce tasks in the inverted index construction. With histogram-based partitioning most chunks are in the range 1100-1300 MB, whereas with fixed-range partitioning the chunk sizes are more widely distributed. This leads to the long tail of task durations seen with fixed-range partitioning.

Workload Distribution in the Distance Matrix Computation

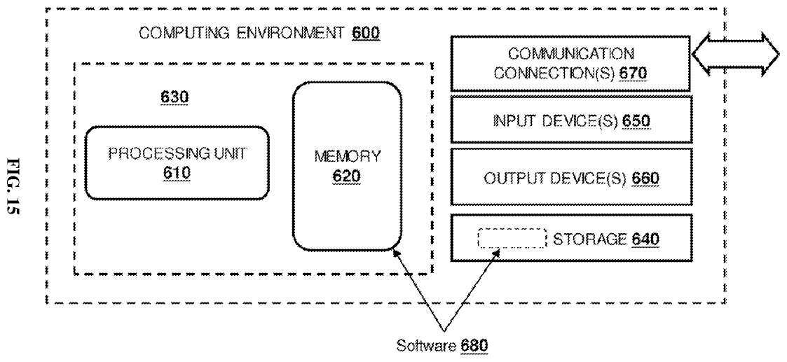

FIG. 14 shows the durations of Map tasks in the distance matrix computation using different input splitting algorithms. Histogram-based input-splitting showed most of Map tasks completed within ±60 seconds from the average while the fixed-range partitioning showed imbalanced durations between Map tasks.

The results show that histogram-based load-balancing is superior to fixed-range load balancing, and importantly reduced the overall duration. Table 9 shows that the using k-mer histograms to load-balance improved overall execution time by more than 10%, even when accounting for the additional time required to construct the histogram.

TABLE 9
Comparison of elapsed times for the
different load-balancing approaches

| Step | Histogram | Fixed-range |
|---|---|---|
| k-mer histogram | 0:08:20 | N/A |
| construction | | |
| Inverted index | 2:38:52 | 3:03:34 |
| construction | | |
| distance matrix | 0:15:36 | 0:17:14 |
| computation | | |
| Total | 3:02:48 | 3:20:47 |

Complete Tara Ocean Viromes Analysis

The complete analysis of ˜4.2 billion reads in the Tara Ocean Viromes (TOV) dataset finished in just less than 19 hours (Table 10). The inverted index construction (k-mer counting) consumed the most time. This is because the shuffle step involves transferring more than 4.7 TB between the Map and Reduce tasks (Table 11, Reduce Shuffle). By comparison, once the inverted indices are constructed, computing the distance matrix (43×43) for the full Tara Ocean Viromes dataset required less than 1.25 hours.

TABLE 10
Elapsed times for Libra based on the
full Tara Ocean Viromes (TOV) dataset

| Step | Elapsed Time |
|---|---|
| k-mer histogram construction | 0:36:36 |
| Inverted index construction | 16:47:06 |
| distance matrix computation | 1:14:44 |
| Total | 18:43:19 |

TABLE 11
I/O during inverted index construction of
the full Tara Ocean Viromes (TOV) dataset

| I/O Type | Size |
|---|---|
| HDFS Read | 552.76 GB |
| HDFS Write | 1323.85 GB |
| Reduce Shuffle | 4761.20 GB |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of metagenome sequence analysis of two or more samples comprising
(i) counting the abundance of each k-mer deconstructed from sequencing reads of nucleic acids in each sample, and
(ii) using a vector space model to compute the genetic distance between each of the two or more samples according to the abundance of the k-mers.

2. The method of claim 1, wherein
(i) counting comprises
(a) constructing a k-mer histogram containing the distribution of k-mers for each sample, and
(b) dividing k-mers into partitions having approximately an equal number of k-mers based on the histogram, preparing an inverted index of the k-mers in each partition and assigning a weight to each k-mer according to its abundance.

3. The method of claim 1, wherein the vector space model comprises assigning each sample a vector, wherein each dimension of each sample's vector corresponds to a unique k-mer and wherein the length and the angle of the vector relates to the abundance of the k-mer and indicates the weight given to the corresponding k-mer in the inverted index.

4. The method of claim 3, wherein the genetic distance between samples is determined using the cosine of the angles between the vectors of the two or more samples.

5. The method of claim 1, wherein the method is computer implemented.

6. The method of claim 5, wherein the method is implemented on a Hadoop platform using Hadoop tasks.

7. A computer implemented method of metagenome sequence analysis of two or more samples comprising
(i) counting the abundance of each k-mer deconstructed from sequencing reads of nucleic acids in each sample comprising
(a) constructing a k-mer histogram containing the distribution of k-mers deconstructed from sequencing reads of nucleic acids in each sample,
(b) dividing k-mers into partitions having approximately an equal number of k-mers based on the histogram, preparing an inverted index of the k-mers in each partition and assigning a weight to each k-mer according to its abundance, and
(ii) using a vector space model to compute the genetic distance between each of the two or more samples according to the abundance of the k-mers, wherein the vector space model comprises assigning each sample a vector, wherein each dimension of each sample's vector corresponds to a unique k-mer and wherein the length and the angle of the vector relates to the abundance of the k-mer and indicates the weight given to the corresponding k-mer in the inverted index,
wherein (i) and (ii) are executed using Map and Reduce task functions on a Hadoop platform comprising a cluster of two or more computers, or
wherein (i) and (ii) are executed using Spark task functions optionally on a Hadoop platform comprising a cluster of two or more computers.

8. The method of claim 7, wherein the genetic distance between samples is determined using the cosine of the angles between the vectors of the two or more samples.

9. The method of claim 7, wherein the counting is distributed over the two or more computers of the Hadoop cluster.

10. The method of claim 7 wherein the sequencing reads are in the form of a set of sample files, each of which contains the sequence data for a single sample.

11. The method of claim 10, wherein the workload across the computers of the cluster is balanced.

12. The method of claim 11, wherein the balancing the workload comprises distributing tasks splitting the files into data blocks at the block boundary.

13. The method of claim 7, wherein the inverted index is indexed by k-mer sequence and comprises a canonical representation of each k-mer, an identifiers of the samples that contain that k-mer, and its frequency in each sample.

14. The method of claim 13, wherein the canonical representation of each k-mer is either the forward form or the reverse complement form of the k-mer depending on which is first alphabetically.

15.-22. (canceled)

23. A method of developing diagnostic information about an infection in a subject comprising
metagenome sequence analysis according the method of claim 1, wherein at least one of the samples is a biological sample from a subject,
determining the taxonomy or a function of the biological sample, and
diagnosing the subject based on the taxonomy or function.

24. A method of prognosing a suspected infection in a subject in need thereof comprising
metagenome sequence analysis according the method of claim 1, wherein at least one of the samples is a biological sample from a subject, and at least one of the samples is a known clinical sample from a clinical subject's infection, and the result or outcome of treatment of the clinical subject is known,
prognosing the subject based on genetic distance between the subject's sample and the clinical sample.

25. The method of claim 23, further comprising providing a treatment to the subject based upon the diagnosis.

26. (canceled)

27. The method of claim 1 wherein (i) and/or (ii) are carried out in real-time.

28. A system for metagenomic analysis, comprising:
one or more processors; and
one or more non-transitory computer readable storage media storing computer readable instructions that when executed by the one or more processors cause the processors to perform the method of any one of claim 1.

29. One or more non-transitory computer-readable media for metagenomics analysis, the non-transitory computer-readable media storing instructions that when executed cause a computer to perform the method of claim 1.
